(19) United States
(12) Patent Application Publication
(10) Pub. No.: US 2019/0101547 A1
(43) Pub. Date: Apr. 4, 2019

(54) COMPOSITIONS AND METHODS FOR IDENTIFYING SUBJECTS AT RISK FOR TRAUMATIC BRAIN INJURY

(71) Applicants: UNIVERSITY OF PITTSBURGH - OF THE COMMONWEALTH SYSTEM OF HIGHER EDUCATION, Pittsburgh, PA (US); AXELA, INC., Toronto (CA)

(72) Inventors: Rachel Berger, Pittsburgh, PA (US); Patrick Kochanek, Pittsburgh, PA (US); Brian J. Pak, Richmond Hill (CA); Paul T. Smith, Acton (CA); Mariya D. Kolesnikova, Toronto (CA); David Frederick Englert, West Hartford, CT (US)

(73) Assignees: UNIVERSITY OF PITTSBURGH - OF THE COMMONWEALTH SYSTEM OF HIGHER EDUCATION, Pittsburgh, PA (US); AXELA, INC., Toronto (CA)

(21) Appl. No.: 16/182,813

(22) Filed: Nov. 7, 2018

Related U.S. Application Data
(63) Continuation of application No. PCT/US2017/032022, filed on May 10, 2017.
(60) Provisional application No. 62/334,345, filed on May 10, 2016.
(60) Provisional application No. 62/483,825, filed on Apr. 10, 2017.

(51) Int. Cl. G01N 33/68

(52) U.S. Cl. CPC G01N 33/6896; G06F 19/24; G01N 2333/70503; G01N 2800/50; G01N 2333/988; G01N 2800/28; G01N 2333/96494

(57) ABSTRACT

The present disclosure relates to compositions and methods for identifying a subject at risk for traumatic brain injury. In particular, the instant disclosure is directed to identification of the levels of the proteins MMP-9 (Matrix Metallopeptidase 9), NSE (Neuron Specific Enolase) and VCAM-1 (Vascular Cell Adhesion Molecule 1) in a subject sample, and correlation of these protein levels with the presence of intracranial injury. In certain embodiments, subject age, gender and hemoglobin level are also correlated with the presence of intracranial injury.

### CROSS-REFERENCE TO RELATED APPLICATIONS

This application is a continuation of International Application No. PCT/US2017/032022, filed May 10, 2017, which claims the benefit of U.S. Provisional Application Ser. No. 62/334,345 filed May 10, 2016, and U.S. Provisional Application Ser. No. 62/483,825 filed Apr. 10, 2017, all of which applications are incorporated herein by reference in their entireties.

### GRANT INFORMATION

This invention was made with government support under grant numbers HD055986 and HD43843 awarded by the National Institutes of Health. The government has certain rights in the invention.

### 1. INTRODUCTION

The present disclosure relates to compositions and methods for identifying a subject at risk for acute traumatic brain injury (TBI). In particular, the instant disclosure is directed to identification of the levels of the proteins MMP-9 (Matrix Metallopeptidase 9), NSE (Neuron Specific Enolase) and VCAM-1 (Vascular Cell Adhesion Molecule 1) in a subject sample, and correlation of these protein levels with the presence of intracranial injury. In certain embodiments, subject age, subject gender and hemoglobin level are also correlated with the presence of intracranial injury

### 2. BACKGROUND

TBI can result in acute intracranial hemorrhage (ICH), which if left untreated, can result in death. Abusive head trauma (AHT)—sometimes referred to as shaken baby syndrome in the lay press—can result in ICH in infants and children, which can subsequently lead to death. AHT is the leading cause of death from traumatic brain injury (TBI) resulting in ICH in children younger than 2 years of age.^{1, 2} Approximately 1 in 3,300 children less than 1 year of age sustains a severe or fatal AHT;² the number who sustain more mild AHT may be 100 times higher.³ Recognition of AHT can be difficult; parents often do not provide a history of trauma, infants present with non-specific symptoms that are seen in a variety of pediatric illnesses, and the physical examination can be normal. In a study published in JAMA in 1999, 31% of infants diagnosed with AHT had been misdiagnosed previously; 4 of the 5 deaths in the study population could have been prevented had the diagnosis of AHT been made earlier.⁴ In a study performed 15 years after the publication in JAMA, the proportion of children with AHT who were initially misdiagnosed was virtually identical, suggesting that education has not been effective at improving early diagnosis (Letson et al, unpublished data). Accurate and timely diagnosis of ICH, and in particular AHT, is of critical importance, not only so that timely treatment can be administered, but because in the case AHT, infants with AHT who are misdiagnosed are likely to return to a violent environment and be injured again or killed. As a result, there is a need for a means to diagnose TBI resulting in ICH, and in particular, AHT, with robust predictive validity and ready availability at the time of acute injury.

### 3. SUMMARY

The present disclosure relates to compositions and methods for identifying a subject at risk for TBI and acute ICH. In particular, the instant disclosure is directed to identification of one or more markers that are present in different concentrations in samples from a well-appearing subject with TBI, compared to samples from age-matched subjects without TBI.

In certain embodiments, the well-appearing subject with TBI and age-matched subjects without TBI are infants. In certain embodiments, the infants are aged 30 days to 1 year.

In certain embodiments, the well-appearing subject with TBI and age-matched subjects without TBI are aged 18 years or younger.

In certain embodiments, the TBI comprises acute ICH.

In certain embodiments, the TBI is AHT.

In certain embodiments, a marker detected according to the methods of the present application is NSE (Neuron Specific Enolase), or a protein activated or inhibited by NSE. In certain embodiments, the concentration of NSE in a sample from a subject with TBI is greater than the concentration of NSE in a sample from a subject without TBI.

In certain embodiments, the marker detected according to the methods of the present application is a protein marker of a physiologic pathway associated with neuronal, glial, and/or axonal injury.

In certain embodiments, the marker detected according to the methods of the present application is MMP-9 (Matrix Metallopeptidase 9), or a protein activated or inhibited by MMP-9. In certain embodiments, the concentration of MMP-9 in a sample from a subject with TBI is greater than the concentration of MMP-9 in a sample from a subject without TBI.

In certain embodiments, the marker detected according to the methods of the present application is a protein marker of a physiologic pathway of blood brain barrier dysfunction, vascular leakage, edema and/or a neuroinflammatory processes associated with wound repair.

In certain embodiments, the marker detected according to the methods of the present application is VCAM-1 (Vascular Cell Adhesion Molecule 1), or a protein activated or inhibited by VCAM-1. In certain embodiments, the concentration of VCAM-1 in a sample from a subject with TBI is less than the concentration of VCAM-1 in a sample from a subject without TBI. In certain embodiments, the concentration of VCAM-1 in a cerebrospinal fluid (CSF) sample from a subject with TBI is greater than the concentration of VCAM-1 in a sample from a subject without TBI.

In certain embodiments, the marker detected according to the methods of the present application is a protein marker of a physiologic pathway of vascular disruption and/or cell adhesion.

In certain embodiments, the marker detected according to the methods of the present application is a protein marker of a metabolic change.

In certain embodiments, markers detected according to the methods of the present application comprise one or more of NSE; MMP-9; VCAM-1; a protein marker of neuronal, glial and/or axonal injury; a protein marker of blood brain barrier dysfunction, vascular leakage, edema and/or a wound repair neuroinflammation; a protein marker of vascular disruption and/or cell adhesion; and/or a protein marker of a metabolic change.

In certain embodiments, markers detected according to the methods of the present application further include blood hemoglobin level of the subject being screened and/or age, and/or gender of the subject. In certain embodiments, the concentration of hemoglobin in a subject with TBI is less than the concentration of hemoglobin in a subject without TBI.

In certain embodiments, protein concentration is determined by measuring protein concentration in a sample from a subject using an apparatus comprising one or more sandwich-type immunoassays, wherein, for example, a first protein binding agent, for example an antibody, is attached to a substrate such as a microsphere, and a second protein binding agent is attached to a detectable marker, such as a fluorescent label.

In certain embodiments, protein and/or hemoglobin concentration is determined by measuring protein and/or hemoglobin concentration in a sample from a subject using an apparatus comprising one or more multiplex immunoassays that can detect a plurality of different markers in a sample, for example, multiplex immunoassay chips, wherein capture agents such as antibodies are printed directly onto the chips. In certain embodiments, the multiplex immunoassay chips are sandwich-type the multiplex immunoassay silicon chips. In certain embodiments, the multiplex immunoassay is a sandwich-type multiplex immunoassay apparatus comprising capture antibodies printed on a TipChip (Axela, Inc., Toronto, ON Canada).

In certain embodiments, the sample is a biological sample, for example, a serum, plasma, CSF, saliva, whole blood, or capillary blood sample.

In certain embodiments, the concentration of NSE in a sample is adjusted to compensate for erythrocyte hemolysis during sample collection, which results in NSE release into the sample, and artificially elevates NSE levels.

In certain embodiments, the level of erythrocyte hemolysis is determined in the sample, for example, by determining the level of hemoglobin in a serum sample, such as by analyzing the serum sample with a HemoCue Plasma Low Hemoglobin Analyzer (HemoCue, Inc. Lake Forest, Calif.; http://www.hemocue.com/) to determine hemoglobin level in the serum sample.

In certain embodiments, the level of erythrocyte hemolysis is determined in the sample, for example, by testing the sample with a sandwich or competitive type immunoassay to determine hemoglobin level in the sample, and/or haptoglobin-hemoglobin complexes, and/or hemopexin-hemoglobin complexes. In certain embodiments, the sandwich-type immunoassay apparatus comprises capture antibodies printed on a TipChip (Axela, Inc., Toronto, ON Canada).

In certain embodiments the level of erythrocyte hemolysis is determined in the sample, for example, by testing the sample using spectrophotometric devices and applying formula accounting for correction of bilirubin, turbidity and other factors. In certain embodiments, the spectrophotometric device is Nanodrop (Thermo Fisher Scientific Inc., Waltham, Mass., USA).

In certain embodiments, the level of erythrocyte hemolysis is determined in the sample, for example, by testing the sample with a sandwich or competitive type immunoassay to determine other markers of hemolysis, such as haptoglobin, hemopexin, bilirubin, ferritin, or lactate dehydrogenase, or any other marker of hemolysis and method of measuring said markers known in the art.

In certain embodiments, a hemoglobin level equal to or greater than 80 mg/dL detected in a subject serum sample requires NSE adjustment.

In certain embodiments, when the NSE concentration detected in a sample requires adjustment, the adjustment can be made according to the following equation:

Adjusted NSE=Unadjusted NSE−(serum hemoglobin level)*(0.077)+4.188

where NSE is in ng/mL, and serum hemoglobin is in mg/dL.

In certain embodiments, a subject is identified as being at risk for TBI when the classification value for the NSE expression level, MMP-9 expression level and/or VCAM-1 expression level in a subject sample, and/or hemoglobin level of the subject, and/or subject age, and/or subject gender is equal to or greater than a threshold cutoff value derived from a Receiver Operator Characteristic (ROC) curve of a classification model of NSE expression level, MMP-9 expression level and/or VCAM-1 expression level in a plurality of control samples, and/or hemoglobin level, and/or age, and/or gender of the control individuals from whom the plurality of control samples were obtained, wherein the threshold cutoff provides for about 80% sensitivity.

In certain embodiments, the threshold cutoff provides for about 90% sensitivity.

In certain embodiments, the classification model is binary logistic regression.

In certain embodiments, the predictor coefficients and cutoff values used in the regression analysis may vary to achieve a set specificity and sensitivity, for example, when using binary logistic regression analysis with cross validation of different data sets.

In certain embodiments, when a subject is identified as being at risk for TBI according to the methods of the present application, the subject is administered a head CT scan and/or a brain magnetic resonance imaging (MRI) scan to diagnose TBI.

In certain embodiments, the present disclosure provides for a kit for detecting the concentration of NSE; MMP-9; VCAM-1; a protein marker of neuronal, glial and/or axonal injury; a protein marker of blood brain barrier dysfunction, vascular leakage, edema and/or a wound repair neuroinflammation; a protein marker of vascular disruption and/or cell adhesion in a sample; and/or a protein marker of metabolic change, wherein the kit comprises one or more capture agents, for example, antibodies, for said proteins, wherein said capture agents are specific for said proteins.

In certain embodiments, said kit comprises reagents to detect other markers, for example hemoglobin, for assessment of red blood cell hemolysis to adjust the concentration of measured markers of TBI to compensate for said hemolysis, for example, said protein markers described herein.

In certain embodiments, said kit comprises a sandwich-type immunoassay, or a multiplex immunoassay, for example, wherein said capture agents are printed directly onto a chip, and processed with suitable buffers, and signal detection reagents to measure the concentration of said proteins in a sample. In certain embodiments, the capture agents are printed on TipChips (Axela, Inc., Toronto, ON Canada) as part of the Ziplex® platform for detecting proteins (Axela, Inc., Toronto, ON Canada).

In certain embodiments, the kit further includes reagents for detecting hemoglobin levels.

In certain embodiments, the present disclosure provides for a kit for detecting NSE, MMP-9, and VCAM-1 levels in a subject sample, wherein the kit comprises

(i) one or more antibodies that are capable of specifically binding to an NSE expression product, MMP-9 expression product, and VCAM-1 expression product;

(ii) immunodetection reagents for the detection of binding of the NSE, MMP-9, and VCAM-1 expression products that had have bound to the corresponding printed antibodies; and

(iii) instructions for determining if the subject is at risk for TBI, comprising
(a) adjusting NSE level to exclude NSE appeared from hemolysis of erythrocytes in the sample;
(b) selecting a threshold value having about 80% or 90% sensitivity for a Receiver Operator Characteristic (ROC) curve of a regression analysis of adjusted NSE, MMP-9, and VCAM-1 expression levels from a plurality of control samples from control individuals with or without TBI; and
(c) identifying the subject as being at risk for TBI when the regression classification value for the NSE, MMP-9, and VCAM-1 expression levels in the subject sample is equal to or greater than the threshold value.

In certain embodiments, the NSE level is adjusted according to the following equation when hemoglobin level is 80 mg/dL or greater in a serum sample from the subject:

Adjusted NSE=Unadjusted NSE−(serum hemoglobin level)*(0.077)+4.188,

where NSE is in ng/mL, and serum hemoglobin in mg/dL.

In certain embodiments, the regression analysis also includes the subject's hemoglobin level. In certain embodiments, the regression analysis includes the subject's age. In certain embodiments, the regression analysis includes the subject's gender. In certain embodiments, the Receiver Operator Characteristic (ROC) curve of a classification model of the plurality of control samples includes hemoglobin levels of the control individuals from whom the plurality of control samples were obtained. In certain embodiments, the classification model of the plurality of control samples includes the age of the control individuals from whom the plurality of control samples were obtained. In certain embodiments, the classification model of the plurality of control samples includes the gender of the control individuals from whom the plurality of control samples was obtained.

### 4. BRIEF DESCRIPTION OF THE FIGURES

FIG. 1 shows a TipChip Array which consist of a 6.5 mm×6.5 mm×350 micron porous silicon chip attached to a polycarbonate tube, as described by Example 1.

FIG. 2 shows the reagent loading configuration for the multiplexed assay described by Example 1.

FIGS. 3A-3C shows a comparison of luminescence intensities of (A) MMP-9, (B) NSE and (C) VCAM-1 measured in five serum samples immediately following collection and after a freeze/thaw cycle as described by Example 1.

FIG. 4 shows receiver operator characteristic (ROC) curves plotted independently for each of the predictor variables MMP-9, adjusted NSE, VCAM-1, age, gender, and hemoglobin for the 578 collected from subjects aged 30 days to 12 months, as described by Example 1.

FIG. 5 shows a receiver operator characteristic (ROC) curve obtained from binary logistic regression analysis using all six predictors (i.e.: MMP-9, adjusted NSE, VCAM-1, hemoglobin, age, gender) from the test subjects aged 30 days to 12 months. Binary logistic regression modeling was performed with twenty independent cross validations.

FIG. 6 shows the formula used for the multivariate model that uses binary logistic regression classification described by Example 1.

FIG. 7 shows the receiver operator characteristic (ROC) curves for the binary logistic regression classification type of the multivariate analysis with different combinations of predictors using the 578 sample set collected from subjects aged 30 days to 12 months old, as described by Example 1.

FIG. 8 shows the scatter plot of inverse logit scores from a logistic regression model for the subjects in the study aged 30 days to 12 months grouped based on CT classification, as described by Example 1.

FIG. 9 shows a flowchart demonstrating the brain abnormalities in the patients in the prospective validation described by Example 2.

FIG. 10 shows a receiver operator characteristic (ROC) curve developed using data from the derivation cohort described by Example 2. The Area Under the Curve was 0.906 (95% CI: 0.893-0.919). Sensitivity and specificity for prediction of abusive head trauma was 95.8% (95% CI: 94.4-97.0) and 54.9% (95% CI: 50.9-58.9) at a cutoff of 0.182. The black line represents the ROC for the entire training set. The gray lines represent the ROC developed for each of the 20-fold cross-validations.

### 5. DETAILED DESCRIPTION

The present disclosure relates to compositions, kits and methods for identifying a subject at risk for TBI. As described herein, a TBI is an injury in which there is some intracranial injury to the brain of a subject, such as acute ICH. A non-limiting example of a TBI includes acute ICH associated with AHT. In certain embodiments, the subject is a child, as described herein, having a mild form of AHT, wherein the child is well-appearing, and therefore not likely to be diagnosed as having AHT using clinical judgment alone.

In particular, the instant application is directed to identification of the proteins, or measuring the levels of, MMP-9, NSE and VCAM-1 in a subject sample, and correlation of these agents with a risk for TBI. In certain embodiments, the compositions, kits and methods of the present application also include measuring the level of hemoglobin in the blood of the subject, and correlating said level with the risk for TBI. In certain embodiments, the correlation further includes the subject's age. In certain embodiments, the correlation further includes the subject's gender.

The present application is based at least in part on the identification of an association between AHT and an increase in NSE concentration, an increase in MMP-9 concentration, and a decrease in VCAM-1 concentration in samples collected from subjects with AHT compared to samples from subjects without AHT, wherein the samples were analyzed using the Ziplex® flow through chip platform (Axela, Inc., Toronto, ON Canada). The present application is also based at least in part on the identification of an association between AHT and a decrease in hemoglobin level in a subject with AHT compared to subjects without AHT.

For example, ROC (Receiver Operator Characteristic) curve analysis of 578 or 599 test subject samples was able to identify threshold values for a logistic regression model for identifying subjects at risk for having AHT with about 90% sensitivity, wherein the specificity of these models ranged from 25 to 67% based on detection of MMP-9, NSE, VCAM-1 and hemoglobin levels in the samples.

An “individual” or “subject” or “patient” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

For clarity and not by way of limitation, this detailed description is divided into the following sub-portions:

5.1 Assay Sampling;

5.2 Methods of screening for subjects at risk for Traumatic Brain Injury; and

5.3 Kits.

### 5.1 Assay Sampling

The present disclosure provides methods, compositions, and kits for identifying a subject at risk for TBI, wherein said TBI is associated with acute ICH, for example, AHT in infants. These methods and compositions will be useful in the diagnosis and treatment of persons with TBI.

In certain embodiments, the present disclosure entails detecting one or more markers such as NSE protein; MMP-9 protein; VCAM-1 protein; a protein marker of neuronal, glial, and/or axonal injury; a protein marker of blood brain barrier dysfunction, vascular leakage, edema and/or a wound repair neuroinflammation; a protein marker of vascular disruption and/or cell adhesion; and/or a protein marker of metabolic changes present in a subject or control sample, for example, through the use of marker binding agents such as antibodies in the immunologic detection of these markers. According to the present disclosure, such detection can be used to detect and quantify the amount of markers, for example, NSE, MMP-9, and/or VCAM-1 protein, present in the subject or control samples.

In certain embodiments, the marker binding agent comprises one or more molecules that have sufficient affinity and specificity for binding to the marker. Examples of marker binding agents include, but are not limited to, aptamers, small molecules, non-antibody proteins and/or peptides (for example, fusion proteins comprising a receptor for a marker described herein), antibodies and/or functional fragments thereof, that have specificity for said markers. Said marker binding agents can further include a detectable marker, such as, for example, a fluorescent or luminescent marker.

Various useful immunodetection methods have been described in the scientific literature, such as, e.g., Nakamura et al. Handbook of Experimental Immunology (4th Ed.), Weir, E., Herzenberg, L. A., Blackwell, C., Herzenberg, L. (Eds.), Vol. 1, Chapter 27, Blackwell Scientific Publ., Oxford, 1987. Immunoassays, in their most simple and direct sense, are binding assays. Such immunoassays include, for example ELISA (enzyme-linked immunosorbent assay) assays.

Although methods may be described in the context of antibody immunoassays, such assays can be practiced using the other marker binding agents described herein.

In general, immunobinding methods include obtaining a biological sample suspected of containing a protein, peptide, antigen or marker, and contacting the sample with an antibody or protein or peptide or marker binding agent under conditions effective to allow the formation of immunocomplexes. In certain embodiments, the antibody or protein or peptide or marker binding agent is immobilized onto a solid surface substrate in accordance with the present disclosure. In certain embodiments, the biological sample includes, but is not limited to, fluids, such as plasma, serum, cerebrospinal fluid, saliva, whole blood, or capillary blood sample.

Contacting a biological sample with the immobilized protein, peptide, antibody, or marker binding agent under conditions effective and for a period of time sufficient to allow the formation of immune complexes (primary immune complexes) generally comprises adding the composition, for example an antibody, to the sample and incubating the mixture for a period of time long enough for the antibodies to form immune complexes with a marker, for example, NSE, MMP-9, and/or VCAM-1 protein. After this time, the marker-antibody mixture will be washed to remove any non-specifically bound marker, e.g., protein/antigens, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, luminescent, biological or enzymatic tags or labels of standard use in the art. U.S. Patents concerning the use of such labels include U.S. Pat. Nos. 3,817,837; 3,850,752; 3,939,350; 3,996,345; 4,277,437; 4,275,149 and 4,366,241, the contents of each of which are incorporated herein by reference. A secondary binding ligand, such as a second antibody or a biotin/streptavidin ligand binding arrangement, as is known in the art, may also be used to detect the antibody-marker immunocomplex.

In certain embodiments, the primary immune complexes may be detected by means of second binding ligands that have binding affinity for the corresponding marker, for example, NSE, MMP-9, and/or VCAM-1 proteins which are bound to protein specific first antibodies (or marker binding agents). In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any unbound or non-specifically bound labeled secondary antibodies or ligands, and the remaining label in the secondary immune complexes is then detected.

In certain embodiments, the detectable label of the secondary antibody is biotin (i.e., the secondary antibody is biotinylated). In certain embodiments, the secondary antibody is detected with a second detectable agent that binds to the biotin, for example, a streptavidin or avidin labeled enzyme, e.g., horseradish peroxidase complex.

Further methods include the detection of primary immune complexes by a two-step approach. A second binding ligand, such as an antibody that has binding affinity for the corresponding marker, for example, NSE, MMP-9, and/or VCAM-1 protein which is bound to the corresponding first antibodies, is used to form secondary immune complexes, as described above. The second binding ligand contains biotin that binds to streptavidin conjugated to an enzyme capable of processing a substrate to a detectable product and, hence, amplifying signal over time. After washing, the secondary immune complexes are contacted with substrate, permitting detection.

In certain embodiments, the present application provides for methods of detecting protein concentration in a sample from a subject using an apparatus comprising one or more substrates, such as chips, wherein the marker binding agents are bound to the substrate, for example, as described by U.S. Pat. Nos. 6,893,816; 7,163,660; 7,470,546; 7,326,561; International PCT Patent Application No. PCT/CA2016/050414, filed Apr. 11, 2016, which published as International Publication No. WO/2016/161524; and International Publication No. WO/2003/004162; each of which is incorporated by reference in its entirety. In other embodiments, the antigen or protein being quantified is attached to the substrate.

In certain embodiments, the present application provides for methods of detecting marker concentration in a sample from a subject using an apparatus comprising one or more sandwich-type immunoassays, wherein a first marker binding agent, for example an antibody, is attached to a substrate such as a microsphere, and a second marker binding agent is attached to a detectable marker, such as a fluorescent or luninescent label.

In certain embodiments, the present application provides for methods of detecting marker concentration in a sample from a subject using an apparatus comprising one or more multiplex immunoassays that can detect a plurality of different markers in a sample. Said multiplex immunoassays include, for example, multiplex immunoassay chips, wherein capture agents are printed directly onto the chips, for example, silicon chips. In certain embodiments, the sandwich-type multiplex immunoassay apparatus comprises capture antibodies printed on a flow through chip (FTC), for example, a TipChip as part of the Ziplex® platform (Axela, Inc., Toronto, ON Canada).

In certain non-limiting embodiments, the FTC multiplex immunoassay provides for detection of the marker concentrations in up to about 5, 10, 15, 20, 25, 30, 35, 40, 45, 50, 55, or 60 minutes.

In certain non-limiting embodiments, the FTC multiplex immunoassay provides for detection of the marker concentrations in a sample volume up to about 0.5, 1, 2, 3, 4, 5, 10, 15, 20, 25, 30, 35, 40, 45, 50, 55, 60, 65, 70, 75, 80, 85, 90, 95, or 100 microliters.

In one non-limiting example, the immunoassay apparatus comprises a square porous silicon chip mounted on a polycarbonate or plastic tube. The chips are made of porous silicon containing a plurality of microchannels per chip (e.g., containing over 200,000 square 10 micron microchannels per chip). An array of capture molecules (e.g., marker binding agents such as antibodies) are printed on the chips wherein the molecules bind to the surface of the microchannels. Within each microchannel is a three dimensional matrix where molecular interactions occur as assay reagents are repeatedly passed back and forth through the channels.

In certain embodiments, the capture molecules are printed on the chips using a print composition comprising capture molecules at a concentration of between about 50 and about 1000 μg/mL, or between about 100 and about 900 μg/mL, or between about 200 and about 800 μg/mL, or between about 300 and about 400 μg/mL.

In certain embodiments, the capture molecules are printed on the chips using a print composition comprising capture antibodies at a concentration of about 400 μg/mL.

In certain embodiments, capture molecules specific for proteins of interest are printed directly onto FTC's, such as TipChips (Axela, Inc., Toronto, ON Canada). During the assay, FTC's such as TipChips are blocked to reduce non-specific interactions, then exposed to a biological sample for capturing marker antigens (for example, MMP-9, NSE and VCAM-1). Next, the FTS's (e.g., TipChips) are incubated with detection antibodies that detect the marker antigen (for example MMP-9, NSE and VCAM-1) bound to its corresponding capture molecule, for example, but not limited to, HRP-conjugated detection antibodies, or biotinylated detection antibodies, then with an amplifying reagent, such as, for example, streptavidin-poly-HRP, that binds to the detection antibodies, and then probed with an assay detection reagent (e.g., HRP substrate) resulting in a detectable signal (e.g., chemiluminescent signal) which is read, for example, by a CCD camera in the apparatus. In certain embodiments, radioactive, colorimetric, luminescent or fluorescent tags may be used as the detectable signal. In certain embodiments, the apparatus and methods of detection are as described in U.S. Pat. Nos. 8,338,189; 8,283,156; 8,076,128; 8,003,060; 7,879,596; 7,314,749; 7,008,794; and 6,981,445; each of which are incorporated by reference in their entireties for all purposes.

In certain embodiments, the present application provides for methods of determining hemoglobin in a subject sample and/or training sample, and/or a means for determining the level of hemolysis in a serum sample obtained from a subject or training individual. Such methods include, but are not limited to spectrophotometric and/or immunoassay measurement of hemoglobin levels, for example, sandwich or competitive type immunoassays, as described herein.

### 5.2 Methods of Screening for Subjects at Risk for Traumatic Brain Injury

According to the present disclosure, one or more markers of TBI, for example, NSE protein; MMP-9 protein; VCAM-1 protein; a protein marker of neuronal, glial, and/or axonal injury; a protein marker of blood brain barrier dysfunction, vascular leakage, edema and/or a wound repair neuroinflammation; a protein marker of vascular disruption and/or cell adhesion; and/or a protein marker of metabolic changes, are present in serum, plasma, CSF, saliva, whole blood, or capillary blood samples from subjects with TBI at concentrations that are different than those in samples from subjects that do not have TBI. Similarly, hemoglobin levels in subjects with TBI are different than hemoglobin levels in subjects without TBI. Detection of these differences in one or more of these markers and/or hemoglobin levels can be used to identify a subject at risk for TBI. While the present disclosure is exemplified in humans, its extension to other species including mammals is contemplated. Assays such as immunoassays using the apparatus described herein, may be used to detect marker levels in a sample. In addition, such analyses may be qualitative or quantitative.

According to the present disclosure, a “subject” or “patient” is a human or non-human animal. Although the animal subject is preferably a human, the concepts, compounds and compositions of the disclosure have application in veterinary medicine as well, e.g., for the treatment of domesticated species, farm animal species, and wild animals or zoological garden animals.

In certain embodiments, the subject is a human subject that is three years old or less, 30 months old or less, 24 months old or less, 18 months old or less, 12 months old or less, 6 months old or less, 3 months old or less, 2 months old or less, or 1 month old or less. In certain embodiments, a human subject is between 0 and about 36 months old, or between 0 and about 24 months old, or between 0 and about 18 months old, or between 0 and about 12 months old, or between 0 and about 6 months old, or between 0 and about 3 months old, or between 0 and about 2 months old, or between 0 and about 1 month old.

In certain embodiments, the subject is a human subject that is 18 years old or less, 17 years old or less, 16 years old or less, 17 years old or less, 16 years old or less, 15 years old or less, 14 years old or less, 13 years old or less, 12 years old or less, 11 years old or less, 10 years old or less, 9 years old or less, 8 years old or less, 7 years old or less, 6 years old or less, 5 years old or less, 4 years old or less, 3 years old or less, 2 years old or less, or 1 years old or less.

In certain embodiments, the subject is a human subject that is greater than 18 years of age.

In certain embodiments, a subject may be a subject suspected of having incurred head trauma, for example (i) a subject manifesting one or more clinical symptom or sign suggestive of head trauma, such as but not limited to a change in level of consciousness or arousal which may be decreased arousal or decreased consciousness, change in sleep pattern (increased sleeping or decreased sleeping), skull deformity, hematoma, anisocoria, motor or sensory deficits, fussiness/irritability, vomiting, seizure-like activity or an apparent life threatening event (sometimes referred to as near-SIDS); or (ii) a subject with a history of head trauma and/or falling.

In certain embodiments, a subject may be a subject suspected of having incurred AHT.

In certain embodiments, a subject may manifest one or more of a change in level of consciousness or arousal which may be decreased arousal or decreased consciousness, change in sleep pattern (increased sleeping or decreased sleeping), skull deformity, hematoma, anisocoria, motor or sensory deficits, fussiness/irritability, vomiting, seizure-like activity or an apparent life threatening event (sometimes referred to as near-SIDS), but not be suspected of having incurred head trauma.

In certain embodiments, a subject may be well-appearing and not be suspected of having incurred head trauma, wherein said well-appearing subject exhibits a Glasgow Coma Scale Score of 13-15 (see, e.g., Teasdale et al., Lancet. 1974 Jul. 13; 2(7872):81-4), or described as well-appearing by a medical doctor, for example, an attending physician.

In humans, the TBI marker and/or hemoglobin disclosed herein may be detected individually or in combination to provide an evaluation of the risk of a subject for TBI. Other markers, such as NSE, MMP-9, and/or VCAM-1 proteins from other species may prove useful, alone or in combination, for similar purposes.

In certain embodiments, the TBI marker detected according to the methods of the present application is alternatively, or can further include, one or more protein markers of a physiologic pathway of neuronal injury (e.g., activation of factors triggering necrosis and/or apoptosis). Examples of such proteins include, but are not limited to, NSE, cleaved tau protein (C-tau), specific breakdown products (SBP), all-Spectrin, phosphorylated neurofilament H (pNF-H), neurofilament H (NF-H), N-methyl-D-aspartate receptor (NMDAR), 70-kDa heat shock proteins (Hsp70), ubiquitin C-terminal hydrolase L1 (UCH-L1), and/or Secretagogin.

In certain embodiments, the TBI marker detected according to the methods of the present application is alternatively, or can further include, one or more protein markers of a physiologic pathway of glial injury (e.g., activation of factors triggering necrosis and/or apoptosis). Examples of such proteins include, but are not limited to, S100β, glial fibrillary acidic protein (GFAP), myelin-basic protein (MBP), C-tau, NMDAR, Hsp70, interleukin 1β (IL-β), interleukin 6 (IL-6), interleukin 8 (IL-8), tumor necrosis factor alpha (TNF-α), and/or aquaporin-4 (AQP4).

In certain embodiments, the TBI marker detected according to the methods of the present application is alternatively, or can further include, one or more protein markers of a physiologic pathway of blood brain barrier dysfunction, vascular leakage, edema (e.g., vasogenic and/or cytotoxic events caused by toxic and inflammatory factors), and/or a neuroinflammatory processes associated with wound repair (e.g., cytokine release and/or cellular stress). Examples of such proteins include, but are not limited to, MMP-9, Hsp70, IL-1β, IL-6, IL-8, vascular endothelial growth factor (VEGF), Claudin-5, von Willebrand factor (vWF), AQP4, TNF-α, and/or interferon gamma (IFN-γ).

In certain embodiments, the TBI marker detected according to the methods of the present application is alternatively, or can further include, one or more protein markers of a physiologic pathway of vascular disruption (e.g., dysregulation of vascular constriction and/or relaxation) and/or cell adhesion. Examples of such proteins include, but are not limited to, VCAM-1, Hsp70, TNF-α, VEGF, Claudin-5, and/or vWF.

In certain embodiments, the TBI marker detected according to the methods of the present application is alternatively, or can further include, one or more protein markers of axonal injury (e.g., mechanical injury and/or neuronal degeneration). Examples of such proteins include, but are not limited to, NSE, S10013, C-tau, MBP, SBP, All-Spectrin, pNF-H, NMDAR, and/or Hsp70.

In certain embodiments, the TBI marker detected according to the methods of the present application is alternatively, or can further include, one or more protein markers of metabolic changes (e.g., hypoxia, altered energy demand, ion homeostasis and neurotransmission, and/or increased repair process). Examples of such proteins include, but are not limited to, Ceruloplasmin and/or hypoxia-inducible factor 1-alpha (HIF-1α).

In certain embodiments, the TBI marker detected according to the methods of the present application is alternatively, or can further include, one or more markers described by Berger et al., “Multiplex assessment of serum biomarker concentrations in well-appearing children with inflicted traumatic brain injury,” Pediatr Res. 2009 January; 65(1):97-102; Gao et al., “Serum amyloid A is increased in children with abusive head trauma: a gel-based proteomic analysis,” Pediatr Res. 2014 September; 76(3):280-6; and/or Mondello et al., “Serum Concentrations of Ubiquitin C-Terminal Hydrolase-L1 and Glial Fibrillary Acidic Protein after Pediatric Traumatic Brain Injury,” Sci Rep. 2016 Jun. 20; 6:28203 (each of which is incorporated by reference in its entirety herein for all purposes), for example, intercellular adhesion molecule (ICAM), VCAM, interleukin 12 (IL-12), eotaxin, tumor necrosis factor (TNF) receptor 2, MMP-9, hepatocyte growth factor (HGF), fibrinogen, IL-6, serum amyloid A, GFAP, and/or UCH-L1.

In certain embodiments, the methods described herein comprise detecting 2, 3, 4, 5, 6, 7, 8, 9, 10 or more of the TBI markers described herein.

In one non-limiting example, the present disclosure provides for methods of determining whether a subject is at risk for TBI comprising detecting an increased level of NSE concentration, detecting an increased level of MMP-9 concentration, and detecting a decreased level of VCAM-1 concentration in a sample from the subject compared to the level of NSE, MMP-9, and VCAM-1 in a sample from a control subject that does not have TBI. In certain embodiments, the method further includes detecting a decreased level of hemoglobin in the subject compared to the hemoglobin level in a subject without TBI. In certain embodiments, the method comprises detecting an increased level of VCAM-1 concentration in a sample from the subject compared to the level of VCAM-1 in a sample from a control subject that does not have TBI when the samples are CSF samples.

In certain embodiments, the samples described herein comprise serum, plasma, cerebrospinal fluid (CSF), saliva, whole blood, or capillary blood. In certain embodiments, the samples described herein comprise serum. In certain embodiments, the samples are diluted with a dilution buffer at a sample:buffer ratio of about 1:200, or about 1:175, or about 1:150, or about 1:125, or about 1:100, or about 1:75, or about 1:50, or about 1:25, or about 1:20, or about 1:15, or about 1:10, or about 1:5, or about 1:1.

In certain embodiments, the methods described herein are optimized to cover clinically relevant ranges of the NSE, MMP-9, and/or VCAM-1 protein markers in serum. The calibration curves for the assay can cover a range from about 10,000 to about 78.1 ng/mL for MMP-9, from about 80 to about 0.6 ng/mL for NSE and from about 3,500 to about 27.3 ng/ml for VCAM-1.

In certain embodiments, a determination of being at risk for TBI is made in a subject when the classification value for a plurality (e.g., 2, 3, 4, 5, or more) TBI marker levels (e.g., NSE expression level, and/or MMP-9 expression level, and/or VCAM-1 expression level) in a subject sample, and/or hemoglobin level in the subject sample, and/or subject age, and/or subject gender is equal to, greater than, or less than a threshold cutoff value.

In certain embodiments the subject classification value and threshold cutoff value are determined using a multivariate statistical model, for example, but not limited to, linear regression, quadratic regression, polynomial regression, logistic regression, support vector machines, linear discriminant analysis, decision trees, or any other multivariate statistical model known in the art for developing algorithms and identifying a threshold value of a data set for a preselected sensitivity and specificity, wherein said algorithm can be used to determine a classification value for any single sample of the data set. In certain embodiments, the threshold cutoff value is determined from the level of a plurality of TBI markers (e.g., 2, 3, 4, 5 or more) from a plurality of training samples from subjects with and/or without TBI, and/or hemoglobin and/or age, and/or gender of the subjects from whom the plurality of training samples were obtained.

In certain embodiments, the threshold cutoff value provides for at least about 60, 65, 70, 75, 80, 85, 90, 95, or 100% sensitivity.

In certain embodiments, the threshold cutoff provides for at least about 20-75%, or about 25-67% specificity. In certain embodiments, the specificity is at least about 20, 25, 30, 35, 40, 45, 50, 55, 60, 65, 70, 75, 80, 85, 90, 95, 99% or more.

In certain embodiments, a subject is identified as being at risk for TBI when a classification value for a plurality (e.g., 2, 3, 4, 5 or more) of TBI marker levels (e.g., NSE expression level, MMP-9 expression level and/or VCAM-1 expression level) in a subject sample, and/or hemoglobin level of the subject, and/or subject age, and/or subject gender is equal to or greater than a threshold cutoff value of a Receiver Operator Characteristic (ROC) curve of a multivariate classification model of said marker levels from a plurality of training samples from subjects with and/or without TBI, and/or hemoglobin and/or age, and/or gender of the subjects from whom the plurality of training samples were obtained, wherein the threshold cutoff provides for about 90% sensitivity.

In certain embodiments, the threshold cutoff of the Receiver Operator Characteristic (ROC) curve of the regression analysis provides for at least about 60, 65, 70, 75, 80, 85, 90, 95, or 100% sensitivity.

In certain embodiments, the threshold cutoff provides for at least about 20-75%, or about 25-67% specificity. In certain embodiments, the specificity is at least about 20, 25, 30, 35, 40, 45, 50, 55, 60, 65, 70, 75, 80, 85, 90, 95, 99% or more.

In certain embodiments, the regression model is a binary logistic regression model.

In certain embodiments, a binary logistic regression classification value for a subject sample can be determined according to the following equation in which the values of the markers have been centered or referenced to the median values of the control samples:

Intercept+KMMP9*[MMP9−median MMP−9]+KadjNSE*[adjusted NSE−median adjusted NSE]+KHb*[Hemoglobin−median Hemoglobin]+KVCAM1*[VCAM1−medianVCAM1]

where ‘median’ represents the median value for the marker (MMP9, adjusted NSE, Hemoglobin, VCAM1) among the controls in a derivation cohort. The coefficients (KMMP9, KadjNSE, KHb, KVCAM1) represent the change per unit increase in the contribution of each of the markers to the calculation of the log odds of a patient being a case. In certain embodiments, MMP-9, NSE and VCAM-1 are measured in nanograms per milliliter (ng/mL) and hemoglobin is measured in grams per deciliter (g/dL).

In certain embodiments, a binary logistic regression classification value for a subject sample can be determined according to the following equation in which the values of the markers have not been centered or referenced to the median values of the control samples:

Intercept+KMMP9*[MMP9]+KadjNSE*[adjusted NSE]+KHb*[Hemoglobin]+KVCAM1*[VCAM1].

In certain embodiments, the threshold cutoff provides for about a 70%, 75%, 80%, 85%, 90%, 95%, or 99% confidence interval of identifying a subject at risk for TBI. In certain embodiments, the subject is not identified as being at risk for TBI when the subject's classification value is within said confidence interval.

In certain embodiments, the NSE levels measured in the assays described herein, and used for determining whether a subject is at risk for TBI, require an additional correction for hemolysis of erythrocytes during sample collection. NSE is present in erythrocytes and hemolysis during sample collection results in its release into serum and therefore artificially elevated NSE levels. Measurement of serum sample hemoglobin can be used to determine the level of hemolysis, and thus be used as a correction factor to determine the quantity of endogenous NSE. For example, an input of the hemoglobin content in serum samples can be determined separately using a HemoCue Plasma Low Hemoglobin Analyzer. NSE levels can be adjusted to compensate for hemolysis in serum samples as described by Berger R. and Richichi R., Ped Crit Care Med 2009; 10:260-263, which is incorporated by reference in its entirety herein.

In certain embodiments, NSE is adjusted when the hemoglobin present in the serum sample is greater than about 40 mg/dL, greater than about 45 mg/dL, greater than about 50 mg/dL, greater than about 55 mg/dL, greater than about 60 mg/dL, greater than about 65 mg/dL, greater than about 70 mg/dL, greater than about 75 mg/dL, greater than about 80 mg/dL, greater than about 85 mg/dL, greater than about 90 mg/dL, greater than about 95 mg/dL, greater than about 100 mg/dL, greater than about 125 mg/dL, greater than about 150 mg/dL, greater than about 175 mg/dL, or greater than about 200 mg/dL.

In certain embodiments, NSE is adjusted when the level of hemoglobin present in the serum sample is greater than or equal to 80 mg/dL.

In certain embodiments, adjusted NSE is calculated according to the following equation:

Adjusted NSE=Unadjusted NSE−(serum hemoglobin level)*(0.077)+4.188

where NSE is measured in ng/mL, and serum hemoglobin in mg/dL.

In certain embodiments, the level of erythrocyte hemolysis is determined in the sample, for example, by testing the sample with a sandwich or competitive type immunoassay to determine hemoglobin level in the sample, and/or haptoglobin-hemoglobin complexes, and/or hemopexin-hemoglobin complexes. (See, e.g., Faulstick et al., “Clearance Kinetics of Haptoglobin-Hemoglobin Complex in the Human.” Blood (1962) 20 (1) 65-71; and Smith et al., “Hemopexin and haptoglobin: allies against heme toxicity from hemoglobin not contenders.,” Frontiers in Physiology, Review, 2015, 6, 187). In certain embodiments, the sandwich-type immunoassay apparatus comprises capture antibodies printed on a TipChip (Axela, Inc., Toronto, ON Canada).

In certain embodiments the level of erythrocyte hemolysis is determined in the sample, for example, by testing the sample using spectrophotometric devices and applying formula accounting for correction of bilirubin, turbidity and other factors. (See, e.g., Tolan et al., “Individualized correction of neuron-specific enolase (NSE) measurement in hemolyzed serum samples,” Clinica Chimica Acta 424 (2013) 216-221). In certain embodiments, the spectrophotometric device is Nanodrop (Thermo Fisher Scientific Inc., Waltham, Mass., USA).

In certain embodiments, the level of erythrocyte hemolysis is determined in the sample, for example, by testing the sample with a sandwich or competitive type immunoassay to determine other markers of hemolysis, such as haptoglobin, hemopexin, bilirubin, ferritin, or lactate dehydrogenase, or any other marker of hemolysis and method of measuring said markers known in the art. (See, e.g., Shih et al., “Haptoglobin testing in hemolysis: Measurement and interpretation,” American Journal of Hematology, 2014, 89 (4), 443-447; Marchand et al., “The predictive value of serum haptoglobin in hemolytic disease,” JAMA 1980; 243:1909-1911; and Barcellini et al., “Clinical Applications of Hemolytic Markers in the Differential Diagnosis and Management of Hemolytic anemia,” Disease Markers, 2015, Article 635670, 7 pages).

In certain embodiments, when a subject is determined to be at risk for TBI, the subject is administered a head CT scan and/or brain MRI to confirm a diagnosis of TBI. In certain embodiments, when a diagnosis of TBI is made, the subject is administered treatment for TBI. In certain embodiments, the treatment comprises an intervention, for example, but not limited to, prevention and/or treatment of a secondary injury, for example, seizures, intubation, paralyzation and sedation, and/or decrease the metabolic needs of the brain (for example, by a medically induced coma).

### 5.3 Kits

In further embodiments, the present application provides kits, such as an immunological kit, for use in detecting one or more TBI markers such as NSE; MMP-9; VCAM-1; a protein marker of neuronal, glial, and/or axonal injury; a protein marker of blood brain barrier dysfunction, vascular leakage, edema and/or a wound repair neuroinflammation; a protein marker of vascular disruption and/or cell adhesion; and/or a protein marker of metabolic changes in a biological sample. Such kits will generally comprise one or more marker binding agents, for example, molecules that have sufficient affinity and specificity for said markers, including, but not limited to, aptamers, small molecules, non-antibody proteins/peptides, antibodies and/or functional fragments thereof, that have specificity for said markers. Said marker binding agents can further include a detectable marker, such as, for example, a fluorescent or luminescent marker.

In certain embodiments, the kit further includes reagents for determining hemoglobin in a subject sample and/or a means for determining the level of hemolysis in a serum sample obtained from a subject.

In certain embodiments, said kit comprises a sandwich-type immunoassay, or a multiplex immunoassay, for example, wherein said capture agents are printed directly onto a chip, and processed with suitable buffers, and signal detection reagents to measure the concentration of said proteins in a sample. In certain embodiments, the capture agents are printed on TipChips (Axela, Inc., Toronto, ON Canada) as part of the Ziplex® platform for detecting proteins (Axela, Inc., Toronto, ON Canada).

In certain embodiments, the immunodetection kits will comprise, in suitable container means, one or more marker binding agents, and one or more ligands, for example capture agents such as antibodies, that bind to marker binding agent-NSE, marker binding agent-MMP-9, and/or marker binding agent-VCAM-1 complexes (e.g., secondary antibodies). In certain embodiments, the one or more ligands (e.g., secondary antibodies) are labeled with a detectable label. In certain embodiments, the kit further includes a means for detecting the ligand (e.g., secondary antibody) with the detectable label.

In certain embodiments, the marker binding agent, e.g., primary antibodies, may be provided bound to a solid support, for example, a sandwich-type immunoassay, or a multiplex immunoassay, such as a multiplex immunoassay chip as described herein. In other embodiment, the marker binding agents are provided bound to a solid support such as a column matrix or well of a microtiter plate. In certain embodiments, the support may be provided as a separate element of the kit.

In certain embodiments, the marker binding agents are not bound to a solid support.

In certain embodiments, the solid support comprises a generally planar porous substrate having opposed surfaces and microchannels extending through a thickness of said substrate, and wherein the one or more marker binding agents are attached to the microchannels.

In certain embodiments, the substrate, for example, the porous substrate, is made of silicon.

In certain embodiments, the substrate, for example, the porous substrate, is manufactured by electrochemical etching of silicon.

In certain embodiments, the substrate, for example, the porous substrate, is manufactured by embossing or molding of a plastic material.

The immunodetection reagents of the kit may include detectable labels that are associated with, or linked to, the given marker binding agent or marker itself. Detectable labels that are associated with or attached to a secondary binding ligand (e.g., an antibody) are also contemplated. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹⁴C, ¹³¹I), enzymes (e.g., alkaline phosphatase, horseradish peroxidase), biotin, avidin, and/or streptavidin, or any other detectable label known in the art.

The kits may further comprise suitable standards of predetermined amounts, including marker binding agents, for example, antibodies, and markers, for example, NSE, MMP-9, and/or VCAM-1 protein. These may be used to prepare a standard curve for a detection assay.

The kits of the disclosure, regardless of type, will generally comprise one or more containers into which the biological agents are placed and, preferably, suitably aliquoted. The components of the kits can be packaged either in aqueous media or in lyophilized form.

The container means of the kits will generally include at least one vial, test tube, flask, bottle, or even syringe or other container means, into which the marker binding agents or marker may be placed, and preferably, suitably aliquoted. Where a second or third binding ligand or additional component is provided, the kit will also generally contain a second, third or other additional container into which this ligand or component may be placed.

The kits of the present disclosure will also typically include a means for containing the control samples, for example, NSE, MMP-9, and/or VCAM-1 protein samples, or marker binding agents, for example, antibodies, and any other reagent containers in close confinement for commercial sale. Such containers may include injection or blow-molded plastic containers into which the desired vials are retained.

In certain embodiments, the kit comprises the Ziplex® immunodetection platform that utilizes TipChip FTC technology (Axela, Inc., Toronto, ON Canada). In certain embodiments, the kit comprises an integrated cartridge as described in International PCT Patent Application No. PCT/CA2016/050414, filed Apr. 11, 2016, which published as International Publication No. WO/2016/161524, which is incorporated by reference in its entirety.

### 6. EXAMPLES

### Example 1: Traumatic Brain Injury (TBI) Assay

The following example describes a blood test that can be used as a screening test for traumatic brain injury (TBI), for example, abusive head trauma (AHT) which is sometimes referred to as shaken baby syndrome and is the leading cause of death from TBI in children younger than 2 years of age. In cases in which this blood test is abnormal, it would prompt a treating physician to consider brain injury and a head computed tomography (CT) or brain magnetic resonance imaging (MRI) scan in an infant in whom the clinician might not ordinarily consider brain injury as an etiology of the infant's symptoms. Any diagnosis based on the abnormality identified on head CT or brain MRI would be made in the same way in which a diagnosis is currently made, based on medical and historical information as well any other corroborating information such as a skeletal survey or dilated eye examination.

### Summary

A multiplexed immunoassay was developed to measure the levels of three serum biomarkers that may assist in identifying which infants are at increased risk of AHT. The concentrations of these markers—matrix metallopeptidase 9 (MMP-9), neuron specific enolase (NSE) and vascular cell adhesion molecule 1 (VCAM-1)—in addition to hemoglobin, age (in months) and gender were analyzed using various multivariate models to develop algorithms that may be used to identify the subset of infants who may benefit from a head CT or brain MRI scan for more definitive diagnosis.

The multiplexed immunoassay for MMP-9, NSE and VCAM-1 were performed on disposable consumables called TipChip Arrays (Axela, Inc., Toronto, ON Canada). These arrays comprise square porous silicon chips mounted on polycarbonate tubes. Capture antibodies were spotted on the silicon chips by pin microarray printing and the chips were then processed for immunoassays comprising sequential incubation of up to eight TipChips in reagents loaded into two separate square-bottomed 96-well plates. Such a process was automated using Axela's Ziplex® instrument.

For multivariate analysis, a sample set consisting of 578 pediatric patients with and without intracranial injury (as defined by head CT, with CT abnormality in cases and normal CT in controls) was used to develop various multivariate models by using a cross validation approach. In addition to biomarker concentrations, multivariate modeling also included patient age, gender and hemoglobin concentrations. The models were cross validated twenty times.

Multivariate analysis was performed by binary step-wise logistic regression using different combinations of predictors (MMP-9, adjusted NSE, VCAM-1, hemoglobin, age and gender). Multivariate models which included hemoglobin significantly outperformed models without hemoglobin. With cut-offs set to achieve assay sensitivity of approximately 90%, the specificity of the best model reached 66%.

### Methods

### TipChip Array Printing

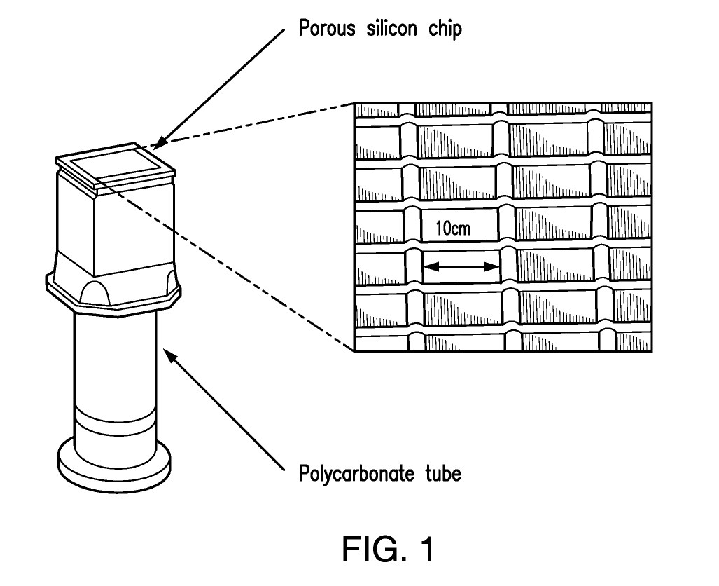

TipChip Arrays consist of 6.5 mm×6.5 mm×350 micron porous silicon chips attached to polycarbonate tubes (FIG. 1). The silicon chips are each composed of over 200,000 10×10 micron microchannels which significantly increases the surface area of the printed spot and facilitates the flow of reagents through the chip when assays are performed. Prior to assembly and printing, the silicon chips are activated by chemical silanization to functionalize the surface with epoxy groups. This allows for the covalent immobilization of amine-containing compounds (e.g.: proteins, antibodies) to the chip surface during printing.

### Printing Methods and Conditions

Array spotting protocols and print buffer constituents were developed to optimize both the activity and long term stability of the deposited capture antibodies. Capture antibodies included anti-MMP-9, anti-VCAM-1 and anti-NSE monoclonal antibodies. Typically TipChip printing was performed at 53% (+7%) relative humidity and at room temperature. Prior to printing, TipChips were placed in the humidified environment for two hours. Capture antibodies were spotted onto the chips by pin microarray printing. After printing, the TipChips were incubated in the humidified atmosphere for a minimum of 12 hours, then placed in a desiccation chamber (<20% relative humidity, room temperature) for four hours. TipChips were packaged into sealed pouches containing desiccant and stored at 4° C. until use.

Each of the printed capture antibodies were acquired commercially and were selected following pairing studies with various secondary antibodies. Extensive checkerboard analyses were performed to optimize antibody printing conditions to maximize antibody activity and stability. This resulted in the optimized antibody print concentration of 400 μg/mL in a printing environment consisting of 1 mg/mL BSA and 5% w/v trehalose in phosphate buffered saline (PBS), pH 7.4. In addition to capture antibodies, fiducials were printed on each TipChip in a preconfigured pattern for spatial orientation of the resulting chemiluminescent image following an assay.

### Multiplexed Immunoassay for MMP-9, NSE and VCAM-1

Sample Set:

Blood samples were collected from infants at Children's Hospital of Pittsburgh of the University of Pittsburgh Medical Center as part of several IRB approved studies which were designed to establish normal serum biomarker concentrations in healthy children without brain injury and to evaluate serum biomarker concentrations in children with TBI of different etiologies (e.g. AHT and accidental TBI). The samples were stored at −80° C. In addition, other blood samples were collected as part of an NIH-funded multi-center study approved by the Children's Hospital of Pittsburgh of the University of Pittsburgh Medical Center IRB, Primary Children's Medical Center IRB (Salt Lake City, Utah) and Lurie Children's Hospital IRB (Chicago, Ill.). As part of this study, infants aged 30 days-364 days who presented to one of the three children's hospitals listed above were eligible for enrollment if they had a temperature of less than 38.3° C., were well appearing, and presented for evaluation of any one of a list of symptoms which are known to be associated with an increased risk of brain injury (e.g. vomiting without diarrhea, fussiness). Children with known brain abnormalities (e.g. hydrocephalus) were not eligible. Children who were not well-appearing and/or presented with a history of trauma were not eligible for enrollment since it would be unlikely that a physician would not consider the possibility of brain injury in these children and that the diagnosis of brain injury would be missed. Blood for both biomarker measurement and hemoglobin was collected by venipuncture either as part of clinical care or specifically for research. Samples were processed by the CLIA-approved hospital laboratory and immediately frozen at −80° C. Hemoglobin was measured in each of the hospitals CLIA-certified labs.

Subjects were determined to be controls or cases based on the results of a head CT and/or brain MRI. Subjects with a normal CT or MRI or those who did not have a CT or MRI completed at the time of enrollment or within 6 months of enrollment or prior to reaching the age of 12 months (whichever came later) were considered to be controls. Subjects with an abnormal head CT were considered to be cases. All CTs and MRIs were evaluated by an attending pediatric neuroradiologist as part of clinical care. All neuroimaging from Salt Lake City and Chicago was de-identified and re-read by the study pediatric neuroradiologist in Pittsburgh. If there was any disagreement between the two interpretations, a study pediatric neurosurgeon in Pittsburgh also evaluated the images. All the abnormal neuroimages performed in Pittsburgh were re-read by the study pediatric neuroradiologist in Pittsburgh. If there was any disagreement between the two interpretations, the study pediatric neurosurgeon in Pittsburgh also evaluated the images. The first 50 normal images in Pittsburgh were re-read by the study pediatric neuroradiologist with 100% concordance. As a result, after the first 50 images, only every 5th normal image was re-read by the study neuroradiologist.

Serum samples were defrosted and aliquoted into 100 μL aliquots and a quantitative assessment of the sample hemoglobin concentration in the sample was performed using a HemoCue Plasma Low Hemoglobin Analyzer (HemoCue, Inc. Lake Forest, Calif.). Samples were then shipped to Axela on dry ice by overnight mail. Samples received at Axela were thawed, aliquoted into multiple 5 μL or 2.5 μL aliquots in 600 μL eppendorf tubes, frozen and stored at −80° C. until use. The hemoglobin measured by the HemoCue Plasma Low Hemoglobin Analyzer results from hemolysis of erythrocytes during sample collection and was measured in mg/dL.

Buffers and Reagents:

A number of different blocking reagents were evaluated. Among these, the blocking agent that provided the lowest background and best signal to noise ratio consisted of 150 mM NaCl, 10 mM NaH₂PO₄, 1 mM EDTA, 0.005% Triton X-100, 1 mg/mL Hammerstein Grade casein, and 0.05% Proclin 300, pH 8.3 (designated as ABR buffer). All washes were performed with 10 mM HEPES, 150 mM NaCl, 0.1% Tween-20, pH 7.3 (HBST). For serum dilutions, a mixture of 95% LowCross-HRP Buffer® (Candor Bioscience) and 5% The Blocking Solution (Candor Bioscience) was used as diluent. The SA-polyHRP (streptavidin-polyHRP) was purchased from Pierce and used at a final concentration of 1 μg/mL in 5 mg/mL BSA in HBST, pH 7.3. SuperSignal West Femto Substrate from Thermo Scientific was used as the chemiluminescent substrate.

Mouse monoclonal capture antibodies, polyclonal biotinylated secondary antibodies and recombinant proteins for MMP-9 and VCAM-1 were purchased from R&D Systems. Mouse monoclonal capture and secondary antibodies for NSE and recombinant NSE were purchased from International Point of Care (IPOC). The NSE secondary antibody was not commercially available as a biotin conjugate and therefore required biotinylation. For biotinylation, the EZ-Link Sulfo-NHS-LC-Biotinylation kit from Thermo Scientific was used according to the manufacturer's instructions. The molar ratio of antibody to biotin used in the reaction was 1:20. The concentration of biotinylated antibodies was determined using a NanoDrop (ND-1000) spectrophotometer.

Assay Development:

The basic structure of the immunoassays for MMP-9, NSE and VCAM-1 consisted of an immobilized mouse monoclonal capture antibody, antigen and biotinylated polyclonal secondary antibodies for MMP-9 and VCAM-1, and biotinylated monoclonal antibody for NSE. A streptavidin poly-horseradish peroxidase (HRP) reagent was used for signal amplification and chemiluminescent detection was utilized for quantification.

Extensive analyses were performed to optimize serum dilution, biotinylated detector antibody concentrations and reagent incubation times separately for each of the three assays to achieve sensitivity and linearity of signal in the clinically relevant concentration ranges. Moreover, a series of assays were performed to determine and minimize “crosstalk” between the components of the individual assays when run in multiplex. This involved the analysis of high levels of two of the biomarkers in multiplex and evaluating the signal on the 3rd biomarker spot. This process was repeated for each of the biomarkers and assay conditions (e.g.: biotinylated secondary antibody concentrations) were modified until the background signal on the 3rd biomarker spot for each protein was virtually undetectable. Based on these analyses, serum dilution was optimized to 1:75 (2 μL in 148 μL serum dilution buffer) and biotinylated secondary antibodies diluted in ABR at 0.25 μg/mL for MMP-9, 0.4 μg/mL for NSE and 0.1 μg/mL for VCAM-1.

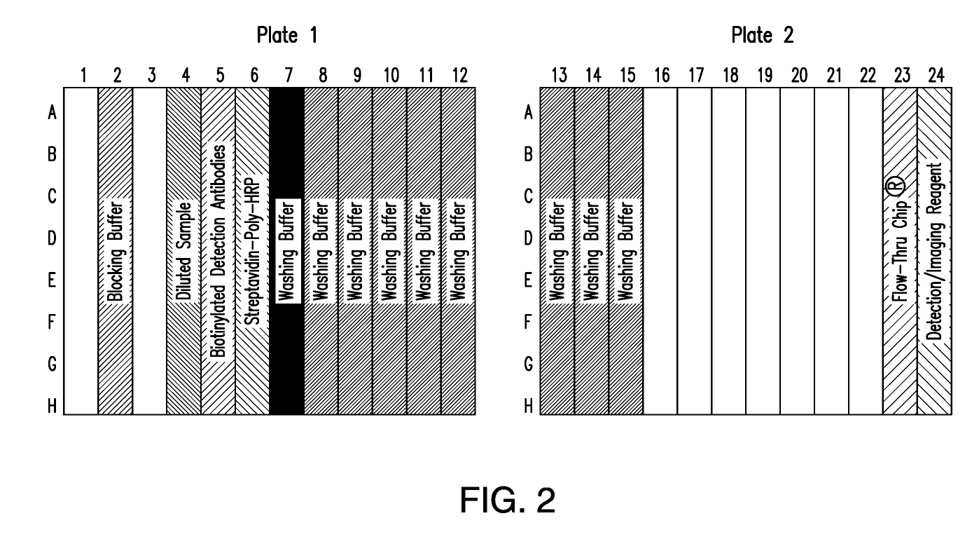

The Ziplex® System automates the sequential incubation of up to eight TipChips in reagents loaded into two separate square-bottomed 96-well plates. The sequence of incubations, duration and mixing frequency are pre-configured by the user. Each chip moves along a single row on the 96-well plates and has access to 24 individual wells. Common reagents are loaded in columns. Reagent loading configuration for the multiplexed assay is shown in FIG. 2. After all of the reagents were loaded into the 96-well plates, TipChips were brought to room temperature, removed from its packaging and loaded into column 23 in the plates. The plates were then loaded into the Ziplex® instrument for assay execution.

The assay protocol was as follows:

1) TipChip leak test—performed to determine if there was a manufacturing defect during TipChip assembly.

### 2) Block in ABR—2 min

3) Incubation in diluted serum—20 min
4) 3× washes in HBST—40 s each
5) Incubation in biotinylated detector antibody—7.5 min
6) 3× washes in HBST—40 s each

### 7) Incubation in SA-polyHRP—7.5 min

8) 3× washes in HBST—40 s each
9) Incubation in chemiluminescent substrate and imaging by CCD camera at 0.1, 1, 10 and 100 s exposure.

All steps were performed with a mixing speed of 4 seconds/cycle.

Serum samples were analyzed at least three times and the average of the replicate measurements was used for biomarker quantitation. An exclusion criterion of median±25% was used to discard outliers in the replicate data.

Calibration Curves:

To account for any variations between wafers or from individual print runs, standard curves for MMP-9, NSE and VCAM-1 were generated for each lot of TipChips printed. Data from samples analyzed on TipChips within the same lot were interpolated from the corresponding calibration curve to determine absolute analyte concentration.

For each protein, 8-point calibration curves were run using 2-fold serial dilutions of recombinant protein. The concentration ranges for each protein were as follows: MMP-9 10,000-78.1 ng/mL; NSE 80-0.6 ng/mL; VCAM-1 3,500-27.3 ng/mL. These ranges covered the clinically relevant concentrations for each protein.

Adjustment of NSE Concentrations:

Due to the presence of NSE in erythrocytes, hemolysis during sample collection can artificially elevate the measurement of serum NSE. As such, the degree of hemolysis was quantified by measuring the levels of hemoglobin in the serum sample using a HemoCue Plasma Low Hemoglobin Analyzer. The process for adjusting NSE is described in Berger R. and Richichi R., Ped. Crit. Care Med. 2009; 10:260-263. NSE adjustments were performed on samples with HemoCue values >80 mg/dL using the following formula:

Adjusted NSE=Unadjusted NSE−(HemoCue)*(0.077)+4.188

where NSE is in ng/mL.

Comparison of Freshly Collected and Frozen Serum Samples:

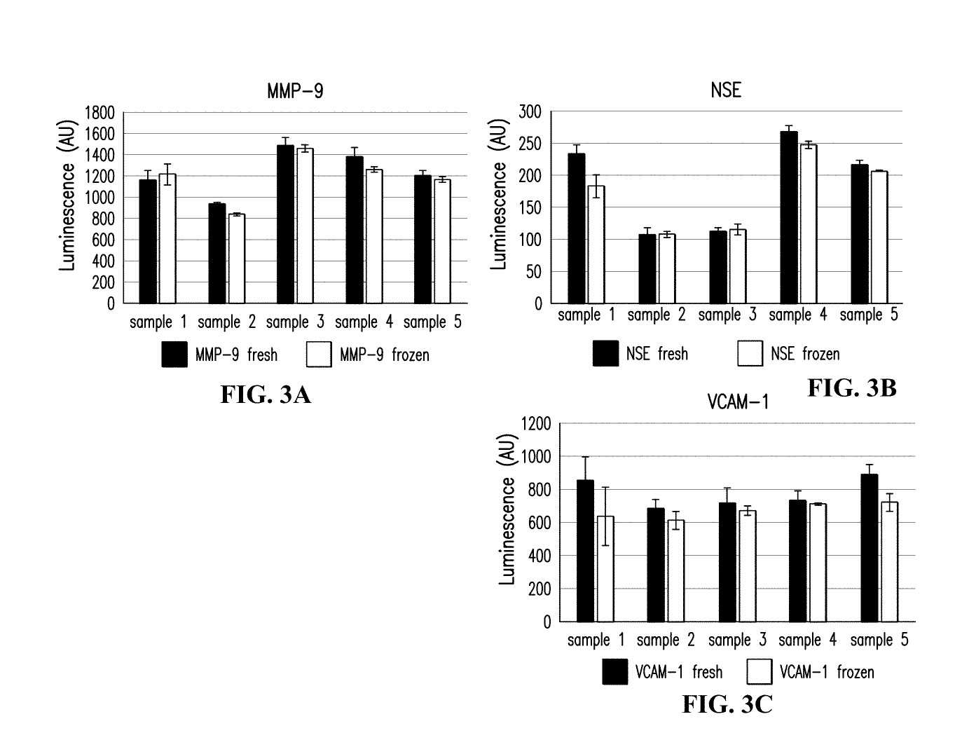

Measurements of the MMP-9, NSE and VCAM-1 biomarkers were performed on previously collected serum samples that had been stored at −80° C. until use. The prospective use of this assay in near patient settings would involve the use freshly acquired serum that do not undergo a freeze/thaw cycle. To determine the applicability of the multivariate models presented herein on freshly acquired serum samples, the impact of serum freeze/thaw on the measurement of MMP-9, NSE and VCAM-1, compared to that measured in fresh samples was investigated. MMP-9, NSE and VCAM-1 levels were measured in five freshly collected serum samples, and measured again in the same samples following storage at −80° C. for several days. The results showed (FIGS. 3A-3C) that the levels of MMP-9, NSE and VCAM-1 did not change after a freeze/thaw cycle, and therefore the multivariate models developed using frozen serum samples are applicable for testing freshly collected serum samples.

### Results

### AHT Assay Data Analysis

Calibration Curve Fitting:

Calibration curve fitting and data interpolation were performed using an online curve fitting software ReaderFit from Hitachi Solutions America (www.readerfit.com). An unweighted 5-parameter logistical fit was used for fitting the MMP-9 and VCAM-1 calibration curves and the concentrations of both proteins were interpolated from their respective curves by the software using the following equation:

F(x)=A+(D/(1+(X/C)^{B})^{E})

A is the MFI/RLU value for the minimum asymptote

B is the Hill slope

C is the concentration at the inflection point

D is the MFI/RLU value for the maximum asymptote

E is the asymmetry factor

X is the luminescent intensity values obtained from the Ziplex assay

For NSE, results showed that the relationship between luminescent intensity and concentration was linear over the relevant concentration range and therefore, an unweighted linear fit was used. Due to the presence of NSE in erythrocytes, hemolysis during sample collection can significantly alter NSE measurement. Therefore, a HemoCue Low Plasma Hemoglobin Analyzer was used to quantify the degree of hemolysis in each sample and NSE levels were adjusted using the formula described previously.

ROC Curve Analysis of Raw Sample Set Data:

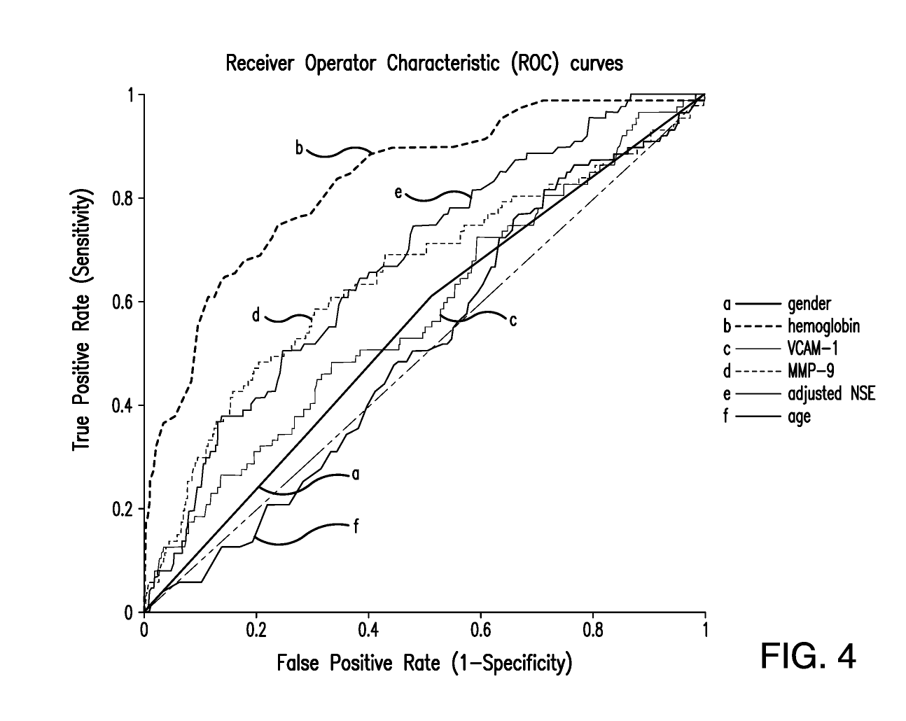

The interpolated concentrations of MMP-9, adjusted NSE and VCAM-1 as well as patient age, gender and hemoglobin levels were determined for a sample set containing serum from 578 subjects aged between 30 days and 12 months. The set included 87 cases (15.1%) with AHT confirmed by CT scans and 491 (84.9%) controls, among which were 274 female (47.4%) and 304 male (52.6%) subjects. Mean (SD) age was 4.5 (3.0) months, and median age 3.5 months. Receiver operator characteristic (ROC) curves were plotted with all 578 samples (FIG. 4). Results of the ROC analysis are summarized in Table 1. Hemoglobin was the best predictor of AHT with an area under the curve (AUC) of 0.83:

### 1. Hemoglobin (AUC=0.83)

### 2. Adjusted NSE (AUC=0.67)

### 3. MMP-9 (AUC=0.65)

### 4. VCAM-1 (AUC=0.58)

### 5. Age (AUC=0.51)

### 6. Gender (AUC=0.55)

Gender had low predictive value in stratifying subjects with AHT with an AUC of 0.55 while age had smaller influence among all tested predictors on distinguishing AHT patients from controls with an AUC of 0.51.

### Multivariate Modeling of Sample Set:

Binary logistic regression was used to develop multivariate models for the classification of AHT and control subjects based on the following six predictors: MMP-9, adjusted NSE, VCAM-1, total serum hemoglobin, age and gender. Binary logistic regression was chosen as one of the methods commonly used for creating non-linear fits to multiple predictors when the outcome is binary. Several other multivariate models including quadratic and support vector machine (SVM) were also tested and their performance was essentially the same as binary logistic regression, therefore they were not further pursued.

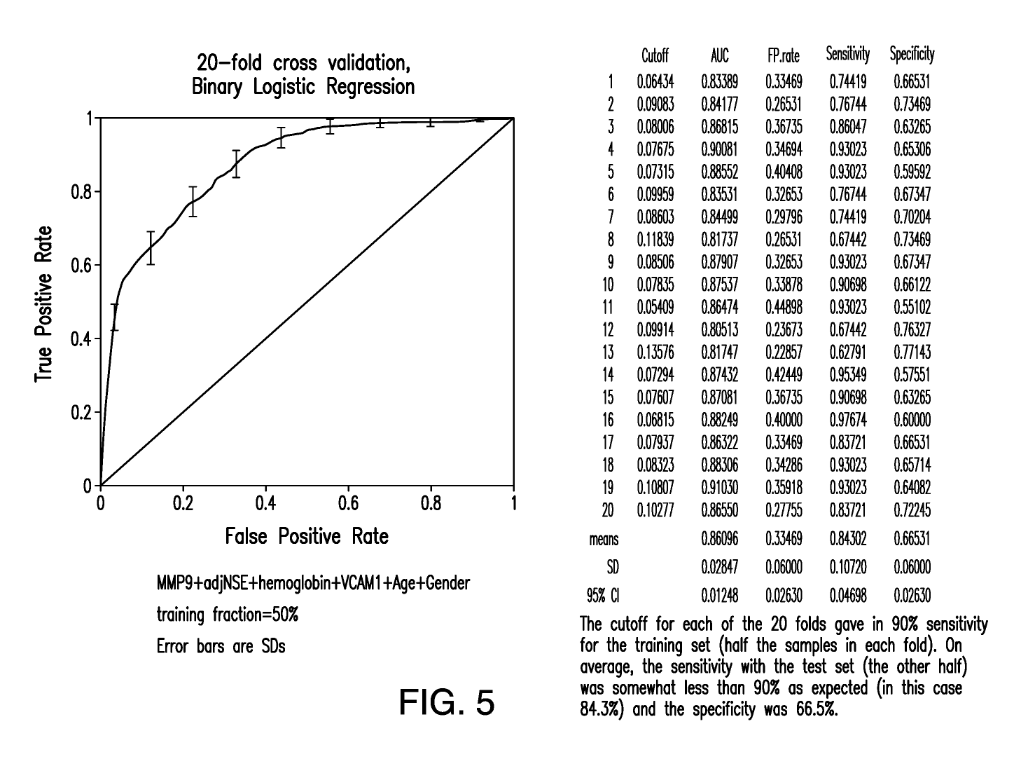

For multivariate modeling, the sample set of 578 samples was taken and randomly divided into two groups containing similar number of controls and cases, one of the sample groups was then used to establish a model and was treated as a training set and another sample group was taken to test the model and used as a test set. Such cross validation of the model was done at least twenty times. The results of the cross validation analysis were used to determine average sensitivity and specificity (FIG. 5).

Different models were developed based on using different subsets of predictors. To assess the performance of the models, cut-offs were set to achieve approximately 90% sensitivity. At these cut-offs, the specificities of the best models reached 66% (Table 1).

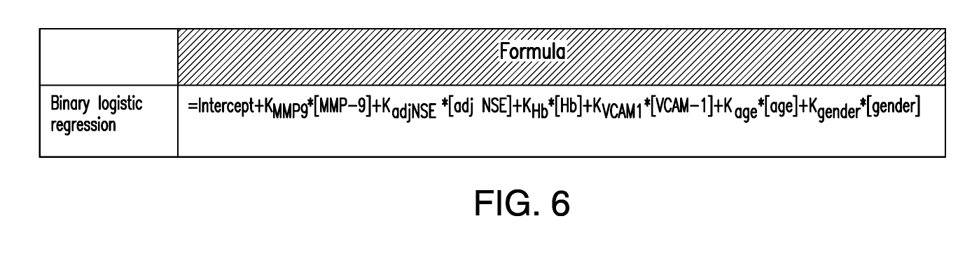

FIG. 6 summarizes the general formula for multivariate models with coefficients (KMMP9, KadjNSE, KserumHb, KVCAM1, Kage, Kgender) for models with different subsets of predictors, including the best model with six predictors MMP-9, adjusted NSE, VCAM-1, hemoglobin, age and gender listed in Table 2.

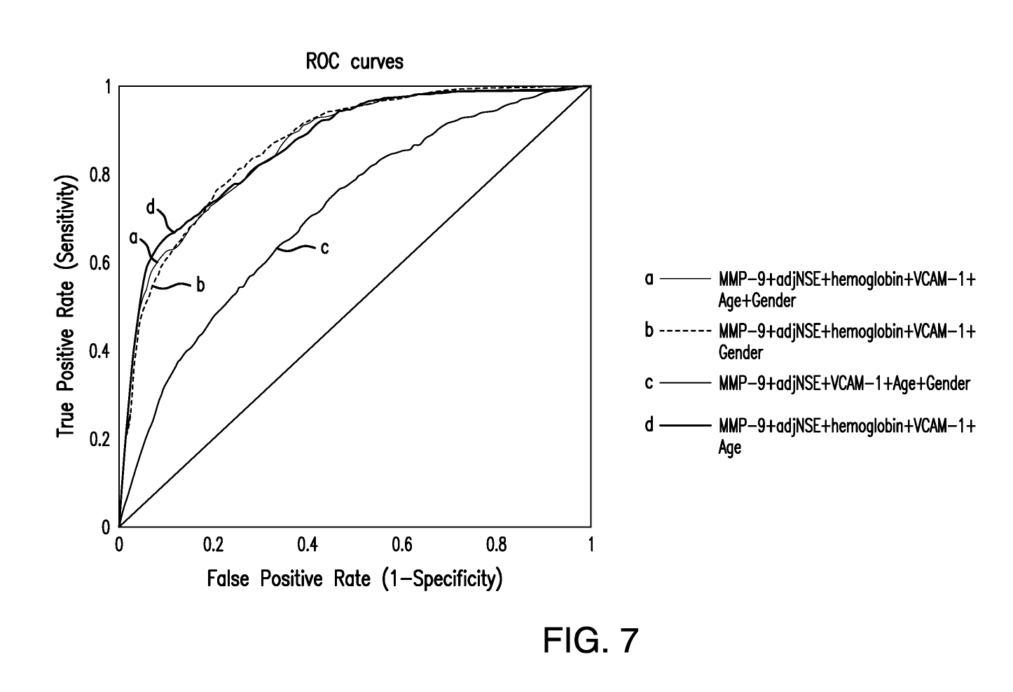

FIG. 7 shows ROC curves for the multivariate models using different predictor combinations. Areas under the curve (AUC) for binary regression analyses conducted with different combination of predictors:

### MMP-9+adjNSE+Hb+VCAM-1+Age+Gender (AUC=0.861)

### MMP-9+adjNSE+Hb+VCAM-1+Age (AUC=0.864)

### MMP-9+adjNSE+Hb+VCAM-1+Gender (AUC=0.864)

### MMP-9+adjNSE+VCAM-1+Age+Gender (AUC=0.710)

TABLE 1
Binary logistic regression multivariate modeling of the 578 subject set using different combinations of predictors.

| | | | Sensitivity | | | | | | FPR | | | |
|---|---|---|---|---|---|---|---|---|---|---|---|---|
| | p values | AUC | | | Sens. | Sens. | Specificity | | | | FPR. | FPR. |
| | avg.fit.p | avg. | avg. | SD. | 95%. | 95%. | avg. | SD. | avg. | SD. | 95%. | 95%. |
| predictors | val | AUC | sensitivity | sensitivity | CI.1 | CI.2 | specificity | specificity | FPR | FPR | CI.1 | CI.2 |
| MMP9 + adjNSE + Hb + VCAM1 + | 0.0000 | 0.864 | 0.860 | 0.070 | 0.830 | 0.890 | 0.666 | 0.057 | 0.334 | 0.057 | 0.309 | 0.359 |
| Gender | | | | | | | | | | | | |
| MMP9 + adjNSE + Hb + VCAM1 + | 0.0000 | 0.861 | 0.843 | 0.107 | 0.796 | 0.890 | 0.665 | 0.060 | 0.335 | 0.060 | 0.309 | 0.361 |
| Age + Gender | | | | | | | | | | | | |
| MMP9 + Hb + VCAM1 + Gender | 0.0000 | 0.861 | 0.855 | 0.067 | 0.826 | 0.884 | 0.656 | 0.046 | 0.344 | 0.046 | 0.324 | 0.364 |
| MMP9 + Hb + VCAM1 + Gender | 0.0000 | 0.858 | 0.859 | 0.077 | 0.825 | 0.893 | 0.653 | 0.057 | 0.347 | 0.057 | 0.322 | 0.372 |
| MMP9 + Hb + VCAM1 + Age + | 0.0000 | 0.861 | 0.871 | 0.065 | 0.843 | 0.899 | 0.652 | 0.048 | 0.348 | 0.048 | 0.327 | 0.369 |
| Gender | | | | | | | | | | | | |
| MMP9 + adjNSE + Hb + VCAM1 + | 0.0000 | 0.864 | 0.856 | 0.066 | 0.827 | 0.885 | 0.643 | 0.060 | 0.357 | 0.060 | 0.331 | 0.383 |
| Age | | | | | | | | | | | | |
| MMP9 + adjNSE + Hb + Age + | 0.0000 | 0.858 | 0.865 | 0.075 | 0.832 | 0.898 | 0.642 | 0.050 | 0.358 | 0.050 | 0.336 | 0.380 |
| Gender | | | | | | | | | | | | |
| MMP9 + adjNSE + Hb + Age | 0.0000 | 0.851 | 0.856 | 0.086 | 0.818 | 0.894 | 0.639 | 0.070 | 0.361 | 0.070 | 0.330 | 0.392 |
| MMP9 + adjNSE + Hb + VCAM1 | 0.0000 | 0.864 | 0.867 | 0.074 | 0.835 | 0.899 | 0.637 | 0.053 | 0.363 | 0.053 | 0.340 | 0.386 |
| MMP9 + adjNSE + Hb + Gender | 0.0000 | 0.864 | 0.895 | 0.084 | 0.858 | 0.932 | 0.633 | 0.059 | 0.367 | 0.059 | 0.341 | 0.393 |
| MMP9 + adjNSE + Hb + Gender | 0.0000 | 0.853 | 0.884 | 0.074 | 0.852 | 0.916 | 0.622 | 0.058 | 0.378 | 0.058 | 0.353 | 0.403 |
| MMP9 + adjNSE + Hb + Age | 0.0000 | 0.858 | 0.893 | 0.047 | 0.872 | 0.914 | 0.616 | 0.046 | 0.384 | 0.046 | 0.364 | 0.404 |
| MMP9 + Hb + Age + Gender | 0.0000 | 0.864 | 0.888 | 0.072 | 0.856 | 0.920 | 0.615 | 0.057 | 0.385 | 0.057 | 0.360 | 0.410 |
| adjNSE + Hb + Age + Gender | 0.0000 | 0.838 | 0.857 | 0.074 | 0.825 | 0.889 | 0.613 | 0.070 | 0.387 | 0.070 | 0.356 | 0.418 |
| MMP9 + adjNSE + Hb + VCAM1 | 0.0000 | 0.863 | 0.885 | 0.061 | 0.858 | 0.912 | 0.613 | 0.044 | 0.387 | 0.044 | 0.368 | 0.406 |
| MMP9 + Hb + VCAM1 + Age | 0.0000 | 0.857 | 0.876 | 0.077 | 0.842 | 0.910 | 0.612 | 0.048 | 0.388 | 0.048 | 0.367 | 0.409 |
| MMP9 + Hb + VCAM1 + Age | 0.0000 | 0.855 | 0.863 | 0.086 | 0.825 | 0.901 | 0.608 | 0.064 | 0.392 | 0.064 | 0.364 | 0.420 |
| MMP9 + Hb + Age + Gender | 0.0000 | 0.865 | 0.892 | 0.062 | 0.865 | 0.919 | 0.594 | 0.059 | 0.406 | 0.059 | 0.380 | 0.432 |
| adjNSE + Hb + Age + Gender | 0.0000 | 0.840 | 0.871 | 0.096 | 0.829 | 0.913 | 0.579 | 0.084 | 0.421 | 0.084 | 0.384 | 0.458 |
| adjNSE + Hb + VCAM1 + Age + | 0.0000 | 0.850 | 0.893 | 0.049 | 0.871 | 0.915 | 0.561 | 0.062 | 0.439 | 0.062 | 0.412 | 0.466 |
| Gender | | | | | | | | | | | | |
| adjNSE + Hb + VCAM1 + Age | 0.0000 | 0.848 | 0.901 | 0.043 | 0.882 | 0.920 | 0.556 | 0.059 | 0.444 | 0.059 | 0.418 | 0.470 |
| Hb + VCAM1 + Age + Gender | 0.0000 | 0.837 | 0.881 | 0.077 | 0.847 | 0.915 | 0.556 | 0.072 | 0.444 | 0.072 | 0.412 | 0.476 |
| adjNSE + Hb + VCAM1 + Gender | 0.0000 | 0.859 | 0.920 | 0.047 | 0.899 | 0.941 | 0.548 | 0.058 | 0.452 | 0.058 | 0.427 | 0.477 |
| adjNSE + Hb + VCAM1 + Gender | 0.0000 | 0.856 | 0.903 | 0.078 | 0.869 | 0.937 | 0.547 | 0.073 | 0.453 | 0.073 | 0.421 | 0.485 |
| adjNSE + Hb + VCAM1 + Age | 0.0000 | 0.852 | 0.900 | 0.056 | 0.875 | 0.925 | 0.546 | 0.094 | 0.454 | 0.094 | 0.413 | 0.495 |
| Hb + VCAM1 + Age + Gender | 0.0000 | 0.845 | 0.885 | 0.047 | 0.864 | 0.906 | 0.544 | 0.058 | 0.456 | 0.058 | 0.430 | 0.482 |
| MMP9 + adjNSE + VCAM1 + Age | 0.0009 | 0.710 | 0.842 | 0.055 | 0.818 | 0.866 | 0.420 | 0.075 | 0.580 | 0.075 | 0.547 | 0.613 |
| MMP9 + adjNSE + VCAM1 + Age | 0.0009 | 0.716 | 0.869 | 0.079 | 0.834 | 0.904 | 0.393 | 0.069 | 0.607 | 0.069 | 0.577 | 0.637 |
| MMP9 + adjNSE + VCAM1 + Age + | 0.0041 | 0.710 | 0.852 | 0.082 | 0.816 | 0.888 | 0.383 | 0.087 | 0.617 | 0.087 | 0.579 | 0.655 |
| Gender | | | | | | | | | | | | |
| MMP9 + adjNSE + VCAM1 + Gender | 0.0087 | 0.684 | 0.857 | 0.063 | 0.829 | 0.885 | 0.374 | 0.051 | 0.626 | 0.051 | 0.604 | 0.648 |
| MMP9 + adjNSE + VCAM1 + Gender | 0.0068 | 0.690 | 0.856 | 0.058 | 0.830 | 0.882 | 0.358 | 0.062 | 0.642 | 0.062 | 0.615 | 0.669 |
| adjNSE + VCAM1 + Age + Gender | 0.0336 | 0.647 | 0.840 | 0.086 | 0.802 | 0.878 | 0.319 | 0.068 | 0.681 | 0.068 | 0.651 | 0.711 |
| MMP9 + VCAM1 + Age + Gender | 0.0019 | 0.673 | 0.844 | 0.086 | 0.806 | 0.882 | 0.313 | 0.081 | 0.687 | 0.081 | 0.651 | 0.723 |
| adjNSE + VCAM1 + Age + Gender | 0.0521 | 0.641 | 0.872 | 0.057 | 0.847 | 0.897 | 0.295 | 0.055 | 0.705 | 0.055 | 0.681 | 0.729 |
| MMP9 + adjNSE + Age + Gender | 0.0261 | 0.662 | 0.852 | 0.076 | 0.819 | 0.885 | 0.258 | 0.070 | 0.742 | 0.070 | 0.711 | 0.773 |
| MMP9 + VCAM1 + Age + Gender | 0.0118 | 0.696 | 0.901 | 0.058 | 0.876 | 0.926 | 0.254 | 0.055 | 0.746 | 0.055 | 0.722 | 0.770 |
| MMP9 + adjNSE + Age + Gender | 0.0194 | 0.690 | 0.898 | 0.068 | 0.868 | 0.928 | 0.213 | 0.065 | 0.787 | 0.065 | 0.759 | 0.815 |

TABLE 2
Binary logistic regression multivariate modeling coefficients for different subsets of predictors.

| Predictors | Intercept | K_{MMP-9} | K_{adj NSE} | K_{Hb} | K_{VCAM-1} | K_{age} | K_{gender} |
|---|---|---|---|---|---|---|---|
| MMP-9 + adjNSE + Hb + | 13.19339 | 0.00041 | 0.03135 | −1.34555 | −0.00323 | 0.00872 | 0.64612 |
| VCAM-1 + Age + Gender | | | | | | | |
| MMP-9 + adjNSE + Hb + | 11.62095 | 0.00047 | 0.02988 | −1.21402 | −0.00305 | 0 | 0.78723 |
| VCAM-1 + Gender | | | | | | | |
| MMP-9 + adjNSE + Hb + | 11.71741 | 0.00044 | 0.02266 | −1.18938 | −0.00255 | −0.017 | 0 |
| VCAM-1 + Age | | | | | | | |

Multivariate Analysis of Blinded Samples from Different Patient Categories:

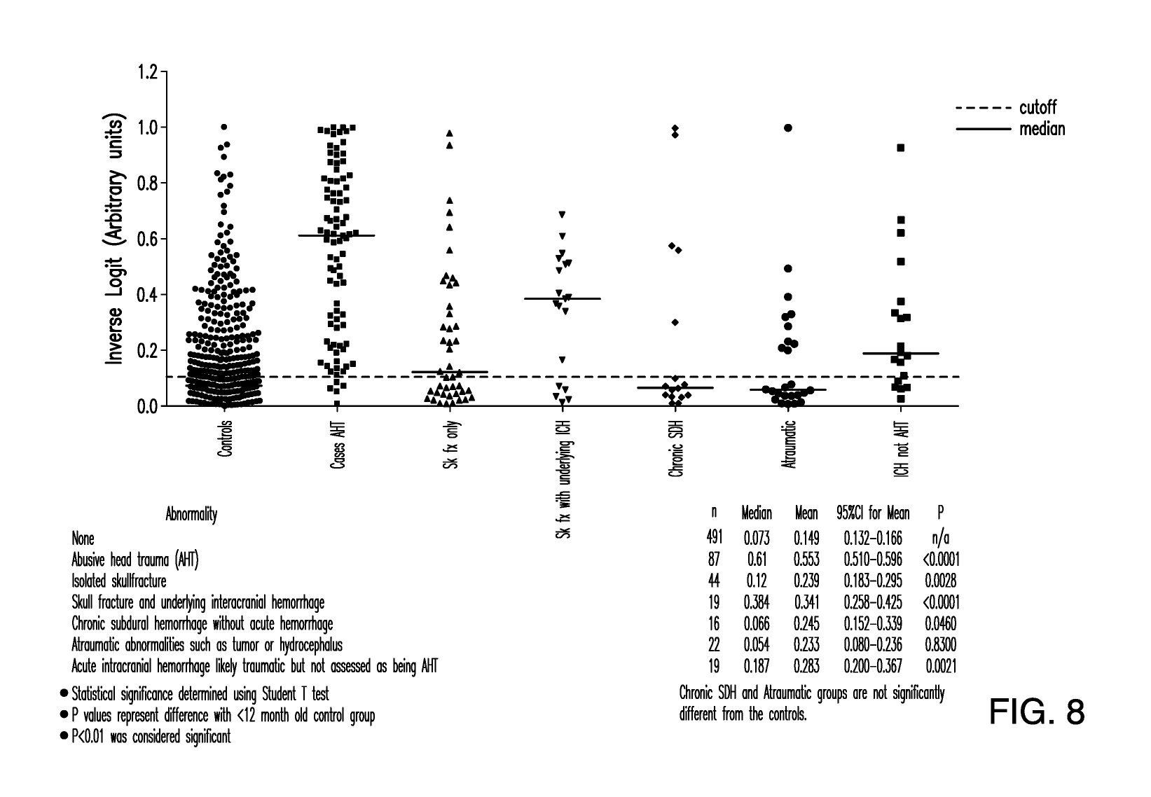

As part of the multi-center study, infants who were ultimately assessed as having brain abnormalities other than AHT were also enrolled. At the time of clinical evaluation, the clinician evaluating the patient would not know if he/she had mild AHT or another brain abnormality which can present with the same signs and symptoms as mild AHT. We, therefore, assessed whether the multivariate model developed to stratify children under 12 months old with AHT would have a similar accuracy if it was tested on children under 12 months old with other brain abnormalities including: Set A (44 subjects)—subjects with isolated skull fracture without underlying brain injury (Sk fx only), Set B (19 subjects)—children with skull fracture and an underlying intracranial hemorrhage (Sk fx with underlying ICH), Set C (16 subjects) included subjects with chronic subdural hemorrhage without acute hemorrhagema (Chronic SDH), Set D (22 subjects)—subjects with atraumatic abnormalities such as tumor or hydrocephalus (Atraumatic), and Set E (19 subjects)—subjects with acute intracranial hemorrhage which likely traumatic but not assessed as being the result of AHT (ICH not AHT) (see table 3 and FIG. 8).

TABLE 3
Application of the binary logistic regression multivariate
model on samples obtained from children under 12 months
old with brain abnormalities other than AHT.

| | Number of | | Test |
|---|---|---|---|
| Set | subjects testing | Cutoff | SENSITIVITY |
| Set A | 44 | 0.135 | 0.477 |
| Set B | 19 | 0.135 | 0.737 |
| Set C | 16 | 0.135 | 0.313 |
| Set D | 22 | 0.135 | 0.318 |
| Set E | 19 | 0.135 | 0.684 |

Predictors: MMP-9 + adjusted NSE + VCAM-1 + hemoglobin + age + gender

The data above suggest that the biomarker panel is most sensitive in children with AHT, but also maintains sensitivity close to 70% in other types of injury in which there is intracranial hemorrhage (Sets B and E). The lower sensitivity in Set B and Set E is likely related to the difference in the physiology of these entities which have only minimal intracranial hemorrhage. Consistent with the expectation that acute hemorrhage is required to have an increase in the serum biomarkers, the sensitivity is very low in children with an isolated skull fracture and no ICH (Set A), children with chronic subdural hemorrhage and no evidence of acute subdural hemorrhage (Set C) and children with atraumatic abnormalities (Set D).

In addition, the multivariate model was tested on samples obtained from children 12 months of age and older to determine applicability of the model to subjects in this age range.

Comparison of the original set of 578 children aged 30 days to 12 months, which included controls and kids with AHT, with subjects having AHT that were aged 12-24 months and subjects having AHT that were over 24 months of age is shown in Table 4.

TABLE 4
Comparison of the multivariate model on samples obtained
from children 12 months of age and older to the 578 children
aged 30 days to 12 months

| | Number of | | | |
|---|---|---|---|---|
| | subjects | | Test | Test |
| Age range | testing | Cutoff | SENSITIVITY | SPECIFICITY |
| 30 days-11.99 | 578 | 0.135 | 0.908 | 0.658 |
| months | | | | |
| 12-23.99 | 25 | 0.135 | 0.923 | 0.583 |
| months | | | | |
| 24 months to | −33 | 0.135 | 0.846 | 0.650 |
| 156 months | | | | |
| 30 days to | 636 | 0.135 | 0.903 | 0.656 |
| 156 months | | | | |

Predictors: MMP-9 + adjusted NSE + VCAM-1 + serum hemoglobin + age + gender

The sensitivity of the model in children 12 months of age and older with mild AHT is comparable to the sensitivity in younger children. The specificity in children greater than 24 months is almost identical to the children below 12 months.

### REFERENCES FOR CITATIONS IN EXAMPLE 1 AND SPECIFICATION

1. Duhaime A C, Christian C W, Rorke L B, et al. Nonaccidental head injury in infants—the “shaken-baby syndrome”. N. Engl J. Med. 1998; 338:1822-1829.
2. Keenan H T, Runyan D K, Marshall S W, et al. A population-based study of inflicted traumatic brain injury in young children. JAMA: The Journal of the American Medical Association 2003; 290:621-626.
3. Theodore A D, Chang J J, Runyan D K, et al. Epidemiologic features of the physical and sexual maltreatment of children in the Carolinas. Pediatrics 2005; 115:e331-337.
4. Jenny C, Hymel K P, Ritzen A, et al. Analysis of missed cases of abusive head trauma. JAMA: The Journal of the American Medical Association 1999; 281:621-626.
5. Berger R P, Pierce M C, Wisniewski S R, et al. Serum S100B concentrations are increased after closed head injury in children: a preliminary study. J. Neurotrauma 2002; 19:1405-1409.
6. Berger R P, Hayes R L, Richichi R, et al. Serum concentrations of ubiquitin C-terminal hydrolase-L1 and alphall-spectrin breakdown product 145 kDa correlate with outcome after pediatric TBI. J. Neurotrauma 2012; 29:162-167.
7. Berger R P, Dulani T, Adelson P D, et al. Identification of inflicted traumatic brain injury in well-appearing infants using serum and cerebrospinal markers: a possible screening tool. Pediatrics 2006; 117:325-332.
8. Berger R P, Adelson P D, Pierce M C, et al. Serum neuron-specific enolase, S100B, and myelin basic protein concentrations after inflicted and noninflicted traumatic brain injury in children. J. of Neurosurgery 2005; 103:61-68.
9. Berger R, Richichi R. Derivation and validation of an equation for adjustment of neuron-specific enolase concentrations in hemolyzed serum. Ped. Crit. Care Med. 2009; 10:260-263.
10. Berger R P, Ta'asan S, Rand A, Lokshin A, Kochanek P. Multiplex assessment of serum biomarker concentrations in well-appearing children with inflicted traumatic brain injury. Pediatr. Res. January 2009; 65:1:97-102.
11. Kuppermann, N., et al. Identification of children at very low risk of clinically-important brain injuries after head trauma: a prospective cohort study. Lancet 2009; 374: 9696: 1160-1170.
12. Nigrovic, L. E., et al. Prevalence of clinically important traumatic brain injuries in children with minor blunt head trauma and isolated severe injury mechanisms. Arch. Pediatr. Adolesc. Med. 2012; 166: 4: 356-361.

### Example 2: Derivation and Validation of a Serum Biomarker Panel to Identify Infants with Acute Intracranial Hemorrhage

### Summary

Abusive head trauma is the leading cause of death due to physical abuse. Missing the diagnosis of abusive head trauma, particularly in its mild form, is common and contributes to increased morbidity and mortality. Serum biomarkers may have potential as quantitative point-of-care screening tools to alert physicians to the possibility of intracranial hemorrhage.

To identify and validate a set of biomarkers that could be the basis of a multivariable model to identify intracranial hemorrhage in well-appearing infants using the Ziplex® System (Axela, Inc., Toronto, ON Canada) a binary logistic regression was used to develop a multivariable model incorporating three serum biomarkers—matrix metallopeptidase-9, neuron-specific enolase and vascular cellular adhesion molecule-1—and one clinical variable—hemoglobin. The model was then prospectively validated. Multiplex biomarker measurements were performed using Flow-Thru™ microarray technology on the Ziplex® System which has the potential as a point-of-care system.

Participants from three pediatric emergency departments in Level I pediatric trauma centers (Children's Hospital of Pittsburgh of University of Pittsburgh Medical Center, Pittsburgh, Pa.; Primary Children's Hospital, Salt Lake City, Utah; and Lurie Children's Hospital, Chicago, Ill.) included well-appearing infants who presented for care due to symptoms which placed them at increased risk of abusive head trauma. The multivariable model—Biomarkers for Infant Brain Injury Score (BIBIS)—was applied prospectively to 599 subjects. The mean (SD) age was 4.7 (3.1) months. Fifty-two percent were boys, 78% were white, and 8% were Hispanic. At a cut-off of 0.182, the model was 89.3% (95% CI: 87.7-90.4) sensitive and 48.0% (95% CI:47.3-48.9) specific for acute intracranial hemorrhage. Positive and negative predictive values were 21.3% and 95.6%, respectively. The model was neither sensitive nor specific for atraumatic brain abnormalities, isolated skull fractures or chronic intracranial hemorrhage.

The present specification discloses a mathematical model which can predict acute intracranial hemorrhage in infants at increased risk of abusive head trauma. The Biomarkers for Infant Brain Injury Score, a multivariable model utilizing three serum biomarker concentrations plus serum hemoglobin can identify infants with acute intracranial hemorrhage. Accurate and timely identification of intracranial hemorrhage in infants without a history of trauma in whom trauma may not be part of the differential diagnosis has the potential to decrease morbidity and mortality from abusive head trauma.

### Methods

### Participants

There were three study groups: the retrospective derivation, the prospective validation and the supplemental cohort of infants with rare intracranial abnormalities.

### Retrospective Derivation

To derive the biomarker-based formula to discriminate infants with and without ICH, serum was used from subjects from an Institutional Review Board (IRB)-approved serum databank at the Safar Center for Resuscitation Research at the University of Pittsburgh.

Serum from subjects in the databank were included in the derivation if they were: age 30-364 days, well-appearing, afebrile (temperature <38.3° C.) and presented to Children's Hospital of Pittsburgh of the University of Pittsburgh Medical Center (CHP) with non-specific symptoms including vomiting or fussiness which can be associated with an increased risk of AHT.^{8,9}

### Prospective Validation

A multicenter prospective validation of the model developed with the derivation cohort was then performed. The study which will be referred to as the parent study was approved by the institutional review boards at Children's Hospital of Pittsburgh of the University of Pittsburgh Medical Center, Pittsburgh, Pa. (CHP), Primary Children's Hospital in Salt Lake City, Utah (SLC) and Ann & Robert H. Lurie Children's Hospital of Chicago (CHG). Written consent was obtained except in cases of suspected abuse, when a waiver of informed consent was approved.

Inclusion criteria are described by Table 5. Enrollment was not consecutive and based on investigator availability. Detailed information about subject classification and data collection has been published previously.²¹

TABLE 5
Study inclusion criteria for the Validation Cohort.

| Inclusion criteria: | |
|---|---|
| All five criteria must be met | Definition |
| 30-364 days of Age^{α} | Self-explanatory |
| Well-appearing | Defined as GCS^{a} score of 13-15 OR by description |
| | of the attending physician when no GCS score |
| | assigned |
| Temperature < 38.3° C. | Defined as no measured temperature ≥ 38.3° C. in |
| | the previous 24 hours |
| No history of trauma | History of trauma not given by caretaker as the |
| | reason for seeking medical care. If history of |
| | trauma was later provided by caretakers, this was |
| | not considered to be a history of trauma for |
| | purposes of eligibility |
| Seeking medical evaluation for one | ALTE as defined by the National Institutes of |
| of the following symptoms: | Health⁴ |
| (1) ALTE/apnea | Vomiting without diarrhea defined as more than |
| (2) Vomiting without diarrhea | four episodes of vomiting in the prior 24 hours or |
| (3) Seizures or seizure-like activity | three or more episodes of vomiting per 24 hours for |
| (4) Soft tissue swelling of the scalp | the prior 48 hours |
| (5) Bruising | |
| (6) Other non-specific neurological | |
| symptom not described above, such | |
| as lethargy, fussiness or poor | |
| feeding | |

Abbreviations:
GCS; Glasgow Coma Scale Score,
ALTE; apparent-life threatening event
^{α}Children <30 days of age were excluded since biomarkers of brain injury can be abnormal in infants <30 days of age due to birth-related trauma.⁴⁴⁻⁴⁶

Supplemental Cohort of Infants with Rare Intracranial Abnormalities

While the overarching goal of the biomarker panel is to identify infants with acute ICH due to AHT, infants with atraumatic intracranial abnormalities, ICH not owing to AHT, chronic ICH or isolated skull fractures can present with similar symptoms as infants with AHT. Therefore, it is critical to assess any biomarker panel in infants with these abnormalities.

Because these abnormalities are rarer than AHT-related ICH,^{22,23} an inadequate number of these subjects in the prospective validation is expected. As a result, serum from infants with these abnormalities were selected from the databank described above and included with prospective validation samples.

Blood was collected as soon as possible after presentation in the emergency department, processed by the hospital laboratory, and de-identified. Serum was stored at −70° C. and shipped to Axela for biomarker analysis. Hemoglobin concentration was measured at the time of enrollment in the hospital laboratory.

### Classification of Patients

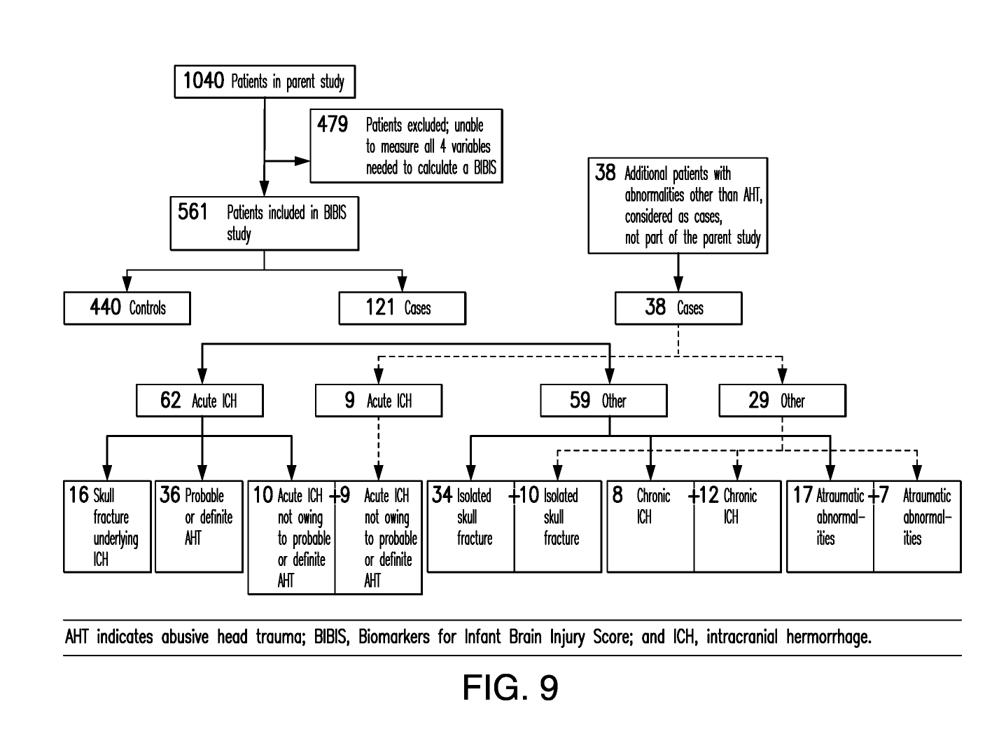

As part of the parent study, subjects were classified as cases or controls based on neuroimaging used a previously published paradigm.²¹ Among cases, brain abnormalities were classified based on etiology (abuse vs not abuse) and type of abnormality (acute ICH not underlying skull fracture, acute ICH underlying skull fracture, chronic ICH, atraumatic abnormality, isolated skull fracture) (FIG. 9).

Computer tomography and magnetic resonance imaging scans were interpreted for clinical care and by a study neuroradiologist. If infants had any acute ICH, they were classified in the acute ICH group. Chronic subdural hemorrhage (SDH) was confirmed with brain MIRI which is standard of care at all three sites.

Abusive head trauma (AHT) was defined as acute ICH and the assessment of probable or definite abuse by each site's hospital-based Child Protection Team (CPT). Using a CPT assessment is a common way to define AHT in clinical research.^{1,9,15} The CPT did not have access to biomarker data.

### Predictors Included in the Model

Based on data from prior publications,^{14-16,14-30} brain specific [neuron-specific enolase (NSE)] and non-brain specific [matrix metallopeptidase-9 (MMP-9), and vascular cellular adhesion molecule-1 (VCAM-1)] markers were included in the model. Three clinical variables (hemoglobin, age and gender) were evaluated for inclusion in the model owing to data suggesting an association with ICH and AHT.^{1,21,31}

### Reagents

Capture and biotinylated reporter antibodies for MMP-9 and VCAM-1 and recombinant protein were obtained from R&D Systems (Minneapolis, Minn.). Antibodies for NSE and recombinant NSE were obtained from International Point of Care (Toronto, ON). EZ-Link Sulfo-NHS-Biotin for biotinylation of NSE reporter antibodies and streptavidin Poly-HRP were obtained from ThermoFisher Scientific (Waltham, Mass.). All other materials and reagents were obtained from Axela Inc. (Toronto, ON).

### Microarray Printing of TipChip Arrays

Capture antibodies were spotted in quadruplicate onto epoxy activated chips using a pin microarray printer.

### Multiplex Immunoassays on the Ziplex System

Multiplex immunoassays were performed on the Ziplex System using 2 L serum and biotinylated secondary antibodies. Following incubation with streptavidin-HRP, biomarkers were detected by chemiluminescence. Biomarker concentrations were taken as the average of replicate sample analyses.

Eight-point calibration curves were generated for each biomarker spanning the clinically relevant concentrations. Five-parameter logistic regression was used for fitting MMP-9 and VCAM-1 calibration curves; linear regression was used for NSE. The serum concentration of each biomarker was interpolated from its respective calibration curve.

Owing to the presence of endogenous NSE in red blood cells, NSE concentrations in serum can be artificially elevated by hemolysis. Therefore, NSE concentrations were adjusted to account for hemolysis using a previously described method.³² Samples with a Hemocue measurement of >500 mg/dL were excluded (to convert to grams per liter, multiply by 10). The technician performing all assays was blinded to clinical data.

### Statistical Analysis

A logistic regression model was fit to the derivation data to identify a prediction model for acute intracranial hemorrhage as a function of biomarkers and covariates. The data for the four continuous-value markers were centered on the median values of the control samples in the derivation set which serve as the baseline for all other samples. A cut-off was chosen so that the sensitivity for the samples in the derivation set was 95%. The model including this cutoff was used for resampling cross validation of the derivation set and for evaluation of the validation set. Binary logistic regression was evaluated with a cross-validation procedure in which the samples in the derivation or validation were divided randomly (resampling without replacement) within twenty folds of approximately equal numbers of resampled derivation and test sets with stratification according to whether samples were cases or controls. The mean and standard deviation area under the curve (AUC) for the Receiver Operating Characteristic (ROC) curves and the specificity and sensitivity, negative and positive predictive for the validation sets from the twenty folds were calculated. Multiple combinations of markers were evaluated (Table 6). A Support Vector Machine (SVM) model was also evaluated using the R SVM function with linear kernel and cost=0.08 (R Programming). A P value of less than 0.05 was considered significant. All P values were 2-sided.

Descriptive statistics were performed using IBM SPSS Version 23.0. Statistical modeling was performed with version R-3.3.2 of the R statistical programming language³³ with the packages ROCR³⁴, e7071³⁵ and openxlsx (R Programming).³⁶ R was used with the RStudio Integrated Development Environment.³⁷

### Results

### Retrospective Derivation

Ninety-nine subjects, 48 with ICH and 51 without ICH, were in the derivation cohort. All 48 subjects with ICH had AHT. Hemoglobin, MMP-9, adjusted NSE, and VCAM-1, but not age or gender, were significantly associated with ICH in the derivation cohort. The model containing hemoglobin plus MMP-9, adjusted NSE and VCAM-1 provided the greatest predictive value (Table 6).

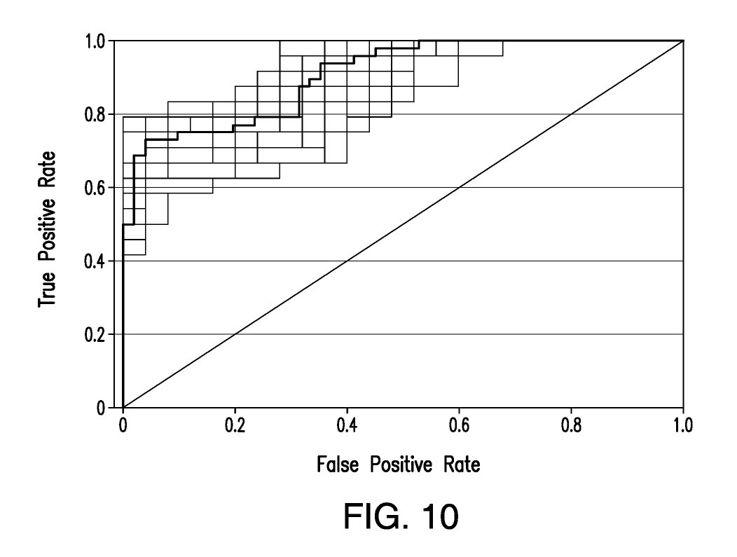

The ROC curve of the binary logistic regression model trained with markers is in FIG. 10. AUC was 0.906 (95% CI: 0.893-0.919). Sensitivity and specificity for prediction of AHT was 95.8% (95% CI: 94.4-97.0) and 54.9% (95% CI: 50.9-58.9) at a cutoff of 0.182. The SVM models provided very similar results with substantial overlap of the confidence intervals from the cross validation (AUC 0.907 (95% CI: 0.894-0.919) with a sensitivity and specificity of 95.8% (95% CI: 94.3-97.4) and 52.9% (95% CI: 47.6-53.0), respectively). Because of the similarity between the models, the SVM model was not pursued further.

TABLE 6
Comparison of binary logistic regression models using
multiple combinations of possible predictors.^{α}

| Markers included in the model | AUC | 95% CI |
|---|---|---|
| Hemoglobin | 0.833 | 0.816-0.850 |
| Hemoglobin: gender | 0.824 | 0.804-0.844 |
| Hemoglobin: age | 0.838 | 0.819-0.857 |
| Hemoglobin: VCAM1 | 0.853 | 0.837-0.869 |
| Hemoglobin: MMP | 0.866 | 0.848-0.884 |
| Hemoglobin: Adjusted NSE | 0.859 | 0.853-0.866 |
| Hemoglobin: VCAM1-MMP | 0.883 | 0.868-0.898 |
| Hemoglobin: VCAM1-Adjusted NSE | 0.890 | 0.877-0.895 |
| Hemoglobin: MMP-Adjusted NSE | 0.884 | 0.877-0.895 |
| Hemoglobin: | 0.903 | 0.891-0.915 |
| MMP-Adjusted NSE-VCAM-1-age-gender | | |
| Hemoglobin: MMP-Adjusted NSE-VCAM-1 | 0.906 | 0.895-0.917 |

Abbreviations:
AUC, area under the curve;
MMP, metallopeptidase-9;
NSE, neuron-specific enolase;
VCAM-1, vascular cellular adhesion molecule-1.
^{α}Models were trained for a sensitivity of about 96%. The combination of hemoglobin, MMP-9, adjusted NSE and VCAM-1 provides the greatest AUC and was, therefore, chosen.

The formula which was developed to classify subjects as cases or controls was:

−2.442+0.000430*[MMP9−median MMP-9]+0.1058*[adjusted NSE−median adjusted NSE]−1.306*[Hemoglobin−median Hemoglobin]0.004165*[VCAM1−medianVCAM1]

where ‘median’ represents the median value for the marker among the controls in the derivation cohort. The coefficients represent the change per unit increase in the contribution of each of the markers relative to the controls to the calculation of the log odds of a patient being a case. MMP-9, NSE and VCAM-1 are measured in nanograms per milliliter (ng/mL) and hemoglobin is measured in grams per deciliter (g/dL). This formula will be referred to as Biomarker of Infant Brain Injury Score (BIBIS).
Prospective Validation Including Infants with Rare Intracranial Abnormalities

There were 1,040 subjects enrolled in the parent study; 54% (561/1040) had BIBIS calculated. Of the 479 subjects in the parent study who could not have BIBIS calculated, the most common reason was that serum was not available for biomarker analysis. Many subjects in the parent study had no blood collected for clinical care and parents did not consent for a blood draw specifically for research. Eighteen subjects were excluded owing to a Hemocue measurement of greater than 0.5 g/dL.³² There was no difference in the mean (SD) age (4.7[3.0] vs 4.7[3.1] months, p=0.51), proportion of boys (52% vs 53%, p=0.80) or the likelihood of being a case patient (22.0% vs 19.4%, p=0.40) in subjects who did and did not have blood available for analysis. A total of 599 subjects (561 from the parent study and 38 with other intracranial abnormalities) had BIBIS calculated (FIG. 9).

The binary logistic regression model was initially evaluated with a validation set which included the 440 controls and the 36 subjects with AHT using the cut-off developed in the derivation set. This was done in order to provide a direct comparison between the derivation and validation sets. The sensitivity and specificity of the cut-off calculated in the derivation set and applied in this cohort was 86.4% (95% CI: 84.1-88.7) and 48.9% (95% CI: 47.9-49.8), respectively.

The binary logistic regression model was then evaluated with a validation set which included the 440 controls and the 71 subjects with acute ICH of any etiology. The sensitivity and specificity of the cut-off in this cohort was 89.3.4% (95% CI: 87.7-90.4) and 48.0% (95% CI:47.3-48.9), respectively. The positive and negative predictive values were 21.3% and 95.6%, respectively. There was substantial overlap with the model characteristics in subjects with AHT compared with those with acute ICH of any etiology, consistent with the modeling predicting acute ICH rather than AHT.

The model was unable to identify abnormalities other than acute ICH. The sensitivity and specificity in the cohort of subjects with atraumatic abnormalities (n=17), chronic ICH (n=8) and isolated skull fracture (n=34) were 52% and 49%, respectively.

### Discussion

This is the first study to use a combination of serum biomarkers and a clinical variable to derive and validate a screening tool which predicts acute ICH in infants at increased risk of AHT. The ability to identify infants who would benefit from neuroimaging has the potential to improve early recognition of AHT and, thereby, decrease morbidity and mortality.

The consistency of the retrospective single-site derivation and the prospective multi-site validation as well as the consistency of the binary logistic regression and SVM models supports the robustness of this approach. The fact that the sensitivity and specificity for acute ICH due to A-IHT and acute ICH from other etiologies is the same is consistent with the fact that that BIBIS does not identify AHT, but rather acute ICH. Maximizing sensitivity rather than accuracy was done because missing AHT has more serious implications than performing neuroimaging in subjects without ICH. This is particularly true if rapid MRI rather than head CT is the diagnostic test performed in subjects with an abnormal BIBIS.^{38,39}

The decision not to re-calculate the intercept of the B BIS model in the validation cohort to account for differences in baseline characteristics of the validation and derivation cohorts was intentional. While there was clearly a difference between the two cohorts given the lower sensitivity of the model in the validation cohort and this difference could have been adjusted for by changing the intercept, the inventors chose not to do this because of the implication for clinical practice. In order to use BIBS for prediction in real-time for a single patient, BIBIS would be calculated in a single patient using a pre-defined formula with a known regression slope and intercept. The intercept cannot be re-calculated in each patient.

When developing a new screening tool, comparison to the current gold standard is important. Since clinical judgment is the current criterion standard for determining which infants with non-specific symptoms should undergo neuroimaging, it is the most relevant comparison. Two studies^{9,11,21} suggest that clinical judgment has a sensitivity of approximately 70%; the data demonstrate that BIBIS is more sensitive.

NSE has been a primary brain-specific biomarker evaluated over a decade.^{14,15,24} MMP-9 and VCAM-1 were identified as potential markers in a study in which 45 potential biomarkers were evaluated using multiplex bead technology.¹⁶ Adult studies also demonstrate increased serum MMP-9 after TBI²⁵⁻²⁷ and implicate it as a marker of hemorrhagic transformation and injury to the blood brain barrier after stroke.²⁸ While MMP-9 and NSE are increased after acute ICH, VCAM-1 is decreased. This is in contrast to cerebrospinal fluid concentrations of VCAM-1 which are increased after pediatric TBI.²⁹ The physiologic basis for the decreased serum VCAM-1 is unknown, but endogenous growth factors may downregulate VCAM-1 in brain microcirculation.³⁰

### CONCLUSION

The inventors have derived and prospectively validated a brain injury score—BIBIS—which uses three serum biomarkers plus hemoglobin to predict which infants are most likely to have acute ICH and would benefit from neuroimaging.

### REFERENCES FOR CITATIONS IN EXAMPLE 2

1. Keenan H T, Runyan D K, Marshall S W, Nocera M A, Merten D F, Sinal S H. A population-based study of inflicted traumatic brain injury in young children. JAMA. 2003; 290(5):621-626.
2. Duhaime A C, Christian C W, Rorke L B, Zimmerman R A. Nonaccidental head injury in infants—the “shaken-baby syndrome”. N Engl J Med. 1998; 338(25): 1822-1829.
3. Parks S, Annest J, Hill A, Karch D. Pediatric Abusive Head Trauma: Recommended Definitions for Public Health Surveillance and Research. Center for Disease Control and Prevention 2012:5.
4. US Department of Health and Human Services AfCaF, Administration on Children, Youth and Families, Children's Bureau. Child Maltreatment 2013. 2013.
5. Flaherty E G. Analysis of caretaker histories in abuse: comparing initial histories with subsequent confessions. Child Abuse Negl. 2006; 30(7):789-798.
6. Haviland J, Russell R I. Outcome after severe non-accidental head injury. Arch Dis Child. 1997; 77(6):504-507.
7. Morris M W, Smith S, Cressman J, Ancheta J. Evaluation of infants with subdural hematoma who lack external evidence of abuse. Pediatrics. 2000; 105(3 Pt 1):549-553.
8. Oral R, Yagmur F, Nashelsky M, Turkmen M, Kirby P. Fatal abusive head trauma cases: consequence of medical staff missing milder forms of physical abuse. Pediatr Emerg Care. 2008; 24(12):816-821.
9. Jenny C, Hymel K P, Ritzen A, Reinert S E, Hay T C. Analysis of missed cases of abusive head trauma. JAMA. 1999; 281(7):621-626.
10. Rubin D M, Christian C W, Bilaniuk L T, Zazyczny K A, Durbin D R. Occult head injury in high-risk abused children. Pediatrics. 2003; 111(6 Pt 1):1382-1386.
11. Letson M M, Cooper J N, Deans K J, et al. Prior opportunities to identify abuse in children with abusive head trauma. Child Abuse Negl. 2016; 60:36-45.
12. Christian C W. The evaluation of suspected child physical abuse. Pediatrics. 2015; 135(5):e1337-1354.
13. Berger R, Hymel K, Gao W. The use of biomarkers after inflicted traumatic brain injury: Insight into etiology, pathophysiology and biochemistry. Clinical Pediatric Emergency Medicine 2006 7(3):186-193.
14. Berger R P, Adelson P D, Richichi R, Kochanek P M. Serum biomarkers after traumatic and hypoxemic brain injuries: insight into the biochemical response of the pediatric brain to inflicted brain injury. Dev Neurosci. 2006; 28(4-5):327-335.
15. Berger R P, Dulani T, Adelson P D, Leventhal J M, Richichi R, Kochanek P M. Identification of inflicted traumatic brain injury in well-appearing infants using serum and cerebrospinal markers: a possible screening tool. Pediatrics. 2006; 117(2):325-332.
16. Berger R P, Ta'asan S, Rand A, Lokshin A, Kochanek P. Multiplex assessment of serum biomarker concentrations in well-appearing children with inflicted traumatic brain injury. Pediatr Res. 2009; 65(1):97-102.
17. Berger R P, Fromkin J, Rubin P, Snyder J, Richichi R, Kochanek P. Serum D-dimer concentrations are increased after pediatric traumatic brain injury. J Pediatr. 2015; 166(2):383-388.
18. Gao W, Lu C, Kochanek P M, Berger R P. Serum amyloid A is increased in children with abusive head trauma: A gel-based proteomic analysis. Pediatr Res. 2014.
19. Berger R P, Houle J F, Hayes R L, Wang K K, Mondello S, Bell M J. Translating biomarkers research to clinical care: applications and issues for rehabilomics. PMR. 2011; 3(6 Suppl 1):S31-38.
20. Hayes R L, Robinson G, Muller U, Wang K K. Translation of neurological biomarkers to clinically relevant platforms. Methods Mol Biol. 2009; 566:303-313.
21. Berger R P, Fromkin J, Herman B, et al. Validation of the Pittsburgh Infant Brain Injury Score for Abusive Head Trauma. Pediatrics. 2016; 138(1).
22. Ostrom Q T, Gittleman H, Liao P, et al. CBTRUS statistical report: primary brain and central nervous system tumors diagnosed in the United States in 2007-2011. Neuro Oncol. 2014; 16 Suppl 4:iv1-63.
23. Game E, Loane M, Addor M C, Boyd P A, Barisic I, Dolk H. Congenital hydrocephalus—prevalence, prenatal diagnosis and outcome of pregnancy in four European regions. Eur J Paediatr Neurol. 2010; 14(2): 150-155.
24. Berger R P, Adelson P D, Pierce M C, Dulani T, Cassidy L D, Kochanek P M. Serum neuron-specific enolase, S100B, and myelin basic protein concentrations after inflicted and noninflicted traumatic brain injury in children. J of Neurosurgery. 2005; 103(1 Suppl):61-68.
25. Grossetete M, Phelps J, Arko L, Yonas H, Rosenberg G A. Elevation of matrix metalloproteinases 3 and 9 in cerebrospinal fluid and blood in patients with severe traumatic brain injury. Neurosurgery. 2009; 65(4):702-708.
26. Suehiro E, Fujisawa H, Akimura T, et al. Increased matrix metalloproteinase-9 in blood in association with activation of interleukin-6 after traumatic brain injury: influence of hypothermic therapy. J Neurotrauma. 2004; 21(12): 1706-1711.
27. Zheng K, Li C, Shan X, et al. Matrix metalloproteinases and their tissue inhibitors in serum and cerebrospinal fluid of patients with moderate and severe traumatic brain injury. Neurol India. 2013; 61 (6):606-609.
28. Turner R J, Sharp F R. Implications of MMP9 for Blood Brain Barrier Disruption and Hemorrhagic Transformation Following Ischemic Stroke. Front Cell Neurosci. 2016; 10:56.
29. Whalen M J, Carlos T M, Kochanek P M, et al. Soluble adhesion molecules in CSF are increased in children with severe head injury. J Neurotrauma. 1998; 15(10):777-787.
30. Wu L, Walas S, Leung W, et al. Neuregulinl-beta decreases IL-1beta-induced neutrophil adhesion to human brain microvascular endothelial cells. Transl Stroke Res. 2015; 6(2):116-124.
31. Berger R P, Fromkin J B, Stutz H, et al. Abusive head trauma during a time of increased unemployment: a multicenter analysis. Pediatrics. 2011; 128(4):637-643.
32. Berger R, Richichi R. Derivation and validation of an equation for adjustment of neuron-specific enolase concentrations in hemolyzed serum. Ped Crit Care Med. 2009; 10(2):260-263.
33. Team R C. A language and environment for statistical computing. R Foundation for Statistical Computing. 2016; https://www.R-project.org/.
34. Sing T, Sander O, Beerenwinkel N, Lengauer T. ROCR: visualizing classifier performance in R. Bioinformatic 2005; 21(20):7881.
35. Meyer D, Dimitriadow E, Hornik K, Weingessel A, Leisch F. Miscellaneous Functions of the Department of Statistics, Probability Theory Group (formerly: E 1071). 2015; R package version 1.6-7.
36. Walker A. Openxlsx: Read, Write and Edit XLSX Files. R Package version 3.0.0. 2015.
37. Team R. RStudio: Integrated Development for R. 2016.
38. Flom L, Fromkin J, Panigrahy A, Tyler-Kabara E, Berger R P. Development of a screening MRI for infants at risk for abusive head trauma. Pediatr Radiol. 2015.
39. Missios S, Quebada P B, Forero J A, et al. Quick-brain magnetic resonance imaging for nonhydrocephalus indications. J Neurosurg Pediatr. 2008; 2(6):438-444.
40. Kumar R G, Boles J A, Wagner A K. Chronic Inflammation After Severe Traumatic Brain Injury: Characterization and Associations With Outcome at 6 and 12 Months Postinjury. J Head Trauma Rehabil. 2015; 30(6):369-381.
41. Mondello S, Gabrielli A, Catani S, et al. Increased levels of serum MAP-2 at 6-months correlate with improved outcome in survivors of severe traumatic brain injury. Brain Inj. 2012; 26(13-14):1629-1635.
42. Wood J N, French B, Fromkin J, et al. Association of Pediatric Abusive Head Trauma Rates With Macroeconomic Indicators. Acad Pediatr. 2016; 16(3):224-232.
43. Apnea and home monitoring: Report of a consensus development conference. Bethesda, M A: U.S. Department of Health and Human Services; 1986. NIH 87-2905.
44. Gazzolo D, Michetti F, Bruschettini M, et al. Pediatric concentrations of S100B protein in blood: age- and sex-related changes. Clin Chem. 2003; 49(6 Pt 1):967-970.
45. Gazzolo D, Marinoni E, di Iorio R, Lituania M, Bruschettini P L, Michetti F. Circulating S100beta protein is increased in intrauterine growth-retarded fetuses. Pediatr Res. 2002; 51(2):215-219.
46. Giuseppe D, Sergio C, Pasqua B, et al. Perinatal asphyxia in preterm neonates leads to serum changes in protein S-100 and neuron specific enolase. Curr Neurovasc Res. 2009; 6(2):110-116.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A kit for detecting expression levels of one or more protein markers selected from the group consisting of NSE, MMP-9, VCAM-1, protein markers of neuronal, glial, and/or axonal injury, protein markers of blood brain barrier dysfunction, vascular leakage, edema and/or wound repair neuroinflammation, protein markers of vascular disruption and/or cell adhesion, and protein markers of metabolic changes in a subject sample, wherein the kit comprises:
(i) one or more agents that are capable of specifically binding to said one or more protein markers;
(ii) reagents for detecting binding of the one or more agents to the one or more protein markers; and
(iii) instructions for determining if the subject is at risk for traumatic brain injury, comprising:
(a) calculating a classification value for the subject based on a classification model of the one or more protein marker levels; and
(b) identifying the subject as being at risk for traumatic brain injury when the classification value is greater than or less than a threshold value.

2. The kit of claim 1, wherein the threshold value corresponds to a predetermined sensitivity level for a multivariate statistical model of the expression levels of a plurality of the protein markers in a plurality of training samples from individuals with traumatic brain injury and control samples from individuals without traumatic brain injury, and
wherein the subject's classification value is calculated using the multivariate statistical model of the expression levels of the plurality of protein markers in the subject sample.

3. The kit of claim 2, wherein the multivariate statistical model is selected from the group consisting of binary logistic regression, linear regression, quadratic regression, polynomial regression, logistic regression, support vector machines, linear discriminant analysis, and decision trees;
the plurality of protein markers comprises NSE, MMP-9, and VCAM-1 expression levels; and/or
the multivariate statistical model of the plurality of protein marker levels further includes hemoglobin levels, individual age and/or individual gender.

4. (canceled)

5. The kit of claim 3, wherein the NSE expression level is adjusted to remove NSE from hemolysis of erythrocytes in the subject sample, training samples from individuals with traumatic brain injury and control samples from individuals without traumatic brain injury.

6. The kit of claim 2, wherein NSE level of the subject sample, training samples from individuals with traumatic brain injury and control samples from individuals without traumatic brain injury are adjusted according to the following equation when hemoglobin level in a serum sample from the subject, individual with traumatic brain injury or individual without traumatic brain injury is 80 mg/dL or greater in the serum sample:
Adjusted NSE=Unadjusted NSE−(serum hemoglobin level)*(0.077)+4.188, where NSE is in ng/mL and serum hemoglobin in mg/dL,
the multivariate statistical model for determining the subject's classification value is a logistic regression model with the following formula in which marker values are referenced to the control group of subjects:
Intercept+KMMP9*[MMP9−median MMP-9]+KadjNSE*[adjusted NSE−median adjusted NSE]+KHb*[Hemoglobin−median Hemoglobin]+KVCAM1*[VCAM1−medianVCAM1] and/or
the multivariate statistical model for determining the subject's classification value is a logistic regression model a logistic regression model with the following formula:
Intercept+KMMP9*[MMP9]+KadjNSE*[adjusted NSE]+KHb*[Hemoglobin]+KVCAM1*[VCAM1].

7.-9. (canceled)

10. The kit of claim 2, wherein the threshold value that is applied to the multivariate model provides at least about 80% sensitivity, or at least about 90% sensitivity; and/or
the subject is not identified as being at risk for traumatic brain injury when the subject's classification value is within a 90% confidence interval of the threshold value.

11.-12. (canceled)

13. The kit of claim 1, wherein the one or more agents that is capable of specifically binding to said one or more protein markers are selected from the group consisting of aptamers, small molecules, non-antibody proteins and/or peptides, antibodies and/or functional fragments thereof, and combinations thereof, and/or
the one or more agents that is capable of specifically binding to said plurality of protein markers are attached to a solid support.

14. (canceled)

15. The kit of claim 2, wherein the subject sample and plurality of training samples from individuals with traumatic brain injury and control samples from individuals without traumatic brain injury are selected from the group consisting of serum, plasma, cerebrospinal fluid, saliva, whole blood, and capillary blood samples; and/or
the subject sample and plurality of training samples from individuals with traumatic brain injury and control samples from individuals without traumatic brain injury are serum.

16. (canceled)

17. The kit of claim 13, wherein the solid support comprises a generally planar porous substrate having opposed surfaces and microchannels extending through a thickness of said substrate, and wherein the one or more agents are attached to the microchannels.

18. The kit of claim 17, wherein the porous substrate is made of silicon;
the porous substrate is manufactured by electrochemical etching of silicon; and/or
the porous substrate is manufactured by embossing or molding of a plastic material.

19.-20. (canceled)

21. A method for identifying a subject at risk for traumatic brain injury comprising
(i) detecting expression levels of one or more protein markers selected from the group consisting of NSE, MMP-9, VCAM-1, protein markers of neuronal, glial, and/or axonal injury, protein markers of blood brain barrier dysfunction, vascular leakage, edema and/or wound repair neuroinflammation, protein markers of vascular disruption and/or cell adhesion, and protein markers of metabolic changes in a subject sample,
(ii) calculating a classification value for the subject based on a classification model of the one or more protein marker levels in the subject sample; and
(iii) identifying the subject as being at risk for traumatic brain injury when the classification value is greater than or less than a threshold value.

22. The method of claim 21, wherein the threshold value corresponds to a predetermined sensitivity level for a multivariate statistical model of the expression levels of a plurality of the protein markers in a plurality of training samples from individuals with traumatic brain injury and control samples from individuals without traumatic brain injury, and
wherein the subject's classification value is calculated using the multivariate statistical model of the expression levels of the plurality of protein markers in the subject sample.

23. The method of claim 22, wherein the multivariate statistical model is selected from the group consisting of binary logistic regression, linear regression, quadratic regression, polynomial regression, logistic regression, support vector machines, linear discriminant analysis, and decision trees;
the plurality of protein markers comprises NSE, MMP-9, and VCAM-1 expression levels, and/or
the multivariate statistical model of the plurality of protein marker levels further includes hemoglobin levels, individual age and/or individual gender.

24. (canceled)

25. The method of claim 23, wherein the NSE expression level is adjusted to remove NSE from hemolysis of erythrocytes in the subject sample, training samples from individuals with traumatic brain injury and control samples from individuals without traumatic brain injury.

26. The method of claim 22, wherein NSE level of the subject sample, training samples from individuals with traumatic brain injury and control samples from individuals without traumatic brain injury are adjusted according to the following equation when hemoglobin level in a serum sample from the subject, individual with traumatic brain injury or individual without traumatic brain injury is 80 mg/dL or greater in the serum sample:
Adjusted NSE=Unadjusted NSE−(serum hemoglobin level)*(0.077)+4.188, where NSE is in ng/mL and serum hemoglobin in mg/dL;
the multivariate statistical model for determining the subject's classification value is a logistic regression model with the following formula in which marker values are referenced to the control group of subjects:
Intercept+KMMP9*[MMP9−median MMP-9]+KadjNSE*[adjusted NSE−median adjusted NSE]+KserumHb*[Hemoglobin−median Hemoglobin]+KVCAM1*[VCAM1−medianVCAM1]; and/or
the multivariate statistical model for determining the subject's classification value is a logistic regression model with the following formula:
Intercept+KMMP9*[MMP9]+KadjNSE*[adjusted NSE]+KHb*[Hemoglobin]+KVCAM1*[VCAM1].

27.-29. (canceled)

30. The method of claim 22, wherein the threshold value that is applied to the multivariate model provides at least about 80% sensitivity, or about 90% sensitivity; and/or
the subject is not identified as being at risk for traumatic brain injury when the subject's classification value is within a 90% confidence interval of the threshold value.

31.-32. (canceled)

33. The method of claim 21, wherein the one or more agents that is capable of specifically binding to said one or more protein markers are selected from the group consisting of aptamers, small molecules, non-antibody proteins and/or peptides, antibodies and/or functional fragments thereof, and combinations thereof; and/or
the one or more agents that is capable of specifically binding to said plurality of protein markers are attached to a solid support.

34. (canceled)

35. The method of claim 22, wherein the subject sample, plurality of training samples from individuals with traumatic brain injury and control samples from individuals without traumatic brain injury are selected from the group consisting of serum, plasma, cerebrospinal fluid, saliva, whole blood, and capillary blood sample; and/or
the subject sample, plurality of training samples from individuals with traumatic brain injury and control samples from individuals without traumatic brain injury are serum.

36. (canceled)

37. The method of claim 33, wherein the solid support comprises a generally planar porous substrate having opposed surfaces and microchannels extending through a thickness of said substrate, and wherein the one or more agents are attached to the microchannels.

38. The method of claim 37, wherein the porous substrate is made of silicon;
the porous substrate is manufactured by electrochemical etching of silicon; and/or
the porous substrate is manufactured by embossing or molding of a plastic material.

39.-40. (canceled)

41. The method of claim 21, wherein the traumatic brain injury comprises acute intracranial hemorrhage.

42. The method of claim 23, wherein hemoglobin levels are measured in the subject sample and plurality of training samples from individuals with traumatic brain injury and control samples from individuals without traumatic brain injury using spectrophotometry or immunoassay.

43. The method of claim 25, wherein the level of erythrocyte hemolysis is determined by measuring the level of haptoglobin-hemoglobin complex, hemopexin-hemoglobin complex, haptoglobin, hemopexin, bilirubin, ferritin, lactate dehydrogenase, or combination thereof in the subject sample and plurality of training samples from individuals with traumatic brain injury and control samples from individuals without traumatic brain injury; and/or
the level of erythrocyte hemolysis is determined by measuring hemoglobin using spectrophotometry or immunoassay.

44. (canceled)
